(19) United States
(12) Patent Application Publication
(10) Pub. No.: US 2009/0191259 A1
(43) Pub. Date: Jul. 30, 2009

(54) Efficient liposomal encapsulation

(75) Inventors: Xingong Li, Robbinsville, NJ (US); Tong Shangguan, Princeton, NJ (US); Alla Polozova, Cranberry, NJ (US); Paul R. Meers, Princeton, NJ (US); Walter R. Perkins, Pennington, NJ (US)

Correspondence Address: FOLEY HOAG, LLP;PATENT GROUP, WORLD TRADE CENTER WEST, 155 SEAPORT BLVD, BOSTON, MA, 02110, US

(73) Assignee: TRANSAVE, INC., MONMOUTH JUNCTION, NJ (US)

(21) Appl. No.: 10/500,932

(22) PCT Filed: Jan. 8, 2003
(86) PCT No.: PCT/US03/00377

§ 371 Date: Jan. 26, 2007

Related U.S. Application Data
(60) Provisional application No. 60/346,287, filed on Jan. 9, 2002.

(51) Int. Cl. A61K 9/127; A61K 31/7088; A61K 38/02; A61K 31/711; A61K 39/00; A61K 39/395

(52) U.S. Cl. 424/450; 514/44; 514/2; 424/184.1; 424/130.1

(57) ABSTRACT

The present invention concerns a method for preparing liposomes, said method comprising the following steps: (I) mixing at least one liposome-forming lipid, a water-miscible organic solvent and aqueous medium Y to form a gel or liquid containing gel particles without sonication; and thereafter (II) (a) mixing the gel or liquid containing gel particles with aqueous medium Z1 to directly form the liposomes; (b) (i) mixing the gel or liquid containing gel particles with aqueous medium Z1 to form a curd or curdy substance; and (ii) mixing the curd or curdy substance with aqueous medium Z2 to directly form the liposomes; or (c) (i) cooling the gel or liquid containing gel particles to form a waxy substance; and (ii) mixing the waxy substance with aqueous medium Z1 to directly form the liposomes; wherein aqueous media Y, Z1 and Z2 are the same or different.

### FIELD OF THE INVENTION

This invention concerns liposomes, methods of preparing liposomes, especially liposomes containing a biologically active substance encapsulated therein, and methods of using the liposomes containing the biologically active substance. The methods of preparing the liposomes of the present invention have the advantages of being simple and able to generate primarily small liposomes of relatively homogeneous particle size with a high entrapment efficiency.

### BACKGROUND OF THE INVENTION

Liposomes are lipid vesicles having at least one aqueous phase completely enclosed by at least one lipid bilayer membrane. Liposomes can be unilamellar or multilamellar. Unilamellar liposomes are liposomes having a single lipid bilayer membrane. Multilamellar liposomes have more than one lipid bilayer membrane with each lipid bilayer membrane separated from the adjacent lipid bilayer membrane by an aqueous layer. The cross sectional view of multilamellar vesicles is often characterized by an onion-like structure.

Liposomes are known to be useful in drug delivery, so many studies have been conducted on the methods of liposome preparation. Descriptions of these methods can be found in numerous reviews (e.g., Szoka et al., “Liposomes: Preparation and Characterization”, in Liposomes: From Physical Structure to Therapeutic Applications, edited by Knight, pp. 51-82, 1981; Deamer et al., “Liposome Preparation: Methods and Mechanisms”, in Liposomes, edited by Ostro, pp. 27-51, 1987; Perkins, “Applications of Liposomes with High Captured Volume”, in Liposomes Rational Design, edited by Janoff, pp. 219-259, 1999).

A method of preparing multilamellar liposome was reported by Bangham et al. (J. Mol. Biol. 13:238-252, 1965). In the method of Bangham et al., phospholipids were mixed with an organic solvent to form a solution. The solution was then evaporated to dryness leaving behind a film of phospholipids on the internal surface of a container. An aqueous medium is added to the container to form multilamellar vesicles (hereinafter referred to as MLVs).

Small unilamellar vesicles (hereinafter referred to as SUVs) were prepared using sonication (Huang, Biochemistry 8:346-352, 1969). A phospholipid was dissolved in an organic solvent to form a solution, which was dried under nitrogen to remove the solvent. An aqueous phase was added to produce a suspension of vesicles. The suspension was sonicated until a clear liquid was obtained, which contained a dispersion of SUVs.

Other methods for the preparation of liposomes were discovered in the 1970s. These methods include the solvent-infusion method, the reverse-phase evaporation method and the detergent removal method. In the solvent-infusion method, a solution of a phospholipid in an organic solvent, most commonly ethanol, was rapidly injected into a larger volume of an aqueous phase under a condition that caused the organic solvent to evaporate. When the organic solvent evaporated upon entry into the aqueous phase, bubbles of the organic solvent's vapor were formed and the phospholipid was left as a thin film at the interface of the aqueous phase and the vapor bubble. As the vapor bubble ascended through the aqueous phase, the phospholipid spontaneously rearranged to form unilamellar and oligolamellar liposomes (e.g., see Batzri et al., Biochim. Biophys. Acta, 298:1015-1019, 1973). Liposomes produced by the solvent-infusion method were mostly unilamellar.

Large unilamellar vesicles (hereinafter referred to as LUVs) were prepared by the reverse-phase evaporation method. In the reverse-phase evaporation method, lipids were dissolved in an organic solvent, such as diethylether, to form a lipid solution. An aqueous phase was added directly into the lipid solution in a ratio of the aqueous phase to the organic solvent of 1:3 to 1:6. The mixture of the lipid/organic solvent/aqueous phase was briefly sonicated to form a homogenous emulsion of inverted micelles. The organic solvent was then removed from the mixture in a two-step procedure, in which the mixture was evaporated at 200-400 mm Hg until the emulsion became a gel, which was then evaporated at 700 mm Hg to remove all the solvent allowing the micelles to coalesce to form a homogeneous dispersion of mainly unilamellar vesicles known as reverse-phase evaporation vesicles (hereinafter referred to as REVs) (e.g., see Papahaduopoulos, U.S. Pat. No. 4,235,871).

In the detergent removal method, a phospholipid was dispersed with a detergent, such as cholate, deoxycholate or Triton X-100, in an aqueous phase to produce a turbid suspension. The suspension was sonicated to become clear as a result of the formation of mixed micelles. The detergent was removed by dialysis or gel filtration to obtain the liposomes in the form of mostly large unilamellar vesicles (e.g., see Enoch et al., Proc. Natl. Acad. Sci. USA, 76:145-149, 1979). The liposomes prepared by the detergent removal method suffer a major disadvantage in the inability to completely remove the detergent, with the residual detergent changing the properties of the lipid bilayer and affecting retention of the aqueous phase contents.

There were also methods for the preparation of large liposomes involving fusion or budding. These methods generally started with liposomes prepared with another method and disrupted the vesicular structures using mechanical or electrical forces. The disruption induced physical strain in the bilayer structure and changed the hydration and/or surface electrostatics. One of the ways of disrupting the existing vesicular structures was by a freezing and thawing process, which produced vesicle rupture and fusion. The freezing and thawing process increased the size and entrapment volume of the liposome.

Fountain et al. (U.S. Pat. No. 4,588,578) described a method for preparing monophasic lipid vesicles (hereinafter referred to as MPVs), which are lipid vesicles having a plurality of lipid bilayers. MPVs are different from MLVs, SUVs, LUVs and REVs. In the method of Fountain et al., a lipid or lipid mixture and an aqueous phase were added to a water-miscible organic solvent in amounts sufficient to form a monophase. The solvent was then evaporated to form a film. An appropriate amount of the aqueous phase was added to suspend the film, and the suspension was agitated to form the MPVs.

Minchey et al. (U.S. Pat. No. 5,415,867) described a modification of the method of Fountain et al. In the method of Minchey et al., a phospholipid, a water-miscible organic solvent, an aqueous phase and a biologically active agent were mixed to form a cloudy mixture. The solvents in the mixture were evaporated, but not to substantial dryness, under a stream of air in a warm water bath at 37° C. until the mixture formed a monophase, i.e., a clear liquid. As solvent removal continued, the mixture became opaque and gelatinous, in which the gel state indicated that the mixture was hydrated. The purging was continued for 5 minutes to further remove the organic solvent. The gelatinous material was briefly heated at 51° C. until the material liquified. The resulting liquid was centrifuged to form lipid vesicles containing the biologically active agent. The aqueous supernatant was removed and the pellet of lipid vesicles was washed several times. The modification of Minchey et al. was that the biologically active agent and the lipid were maintained as hydrated at all times to avoid the formation of a film of the biologically active agent and lipid upon the complete removal of all the aqueous phase. During evaporation of the organic solvent, the presence of a gel indicated that the monophase was hydrated.

Different techniques were developed to improve the encapsulation efficiency for biologically active compounds. However, little progress has been made to conveniently and efficiently encapsulate molecules, especially large molecules, into small or medium sized liposomes or to devise liposome production to make liposomes of a relatively homogeneous size distribution without resorting to size reduction methodologies (e.g. extrusion and homogenization). The prior art methods of preparing liposomes suffer from some or all of the following problems: being time consuming and not economical, having a low entrapment efficiency and/or generating vesicles of heterogenous size distribution requiring sonication or extrusion to remove large vesicles. An improved method of preparing liposomes is needed. The present invention has solved the problems by presenting a new relatively simple method of making liposomes having a high entrapment efficiency and of relatively homogeneous size.

### SUMMARY OF THE INVENTION

The present invention involves the formation of liposomes via the hydration of a gel or liquid containing gel particles, wherein the gel or the liquid containing gel particles comprises at least one liposome-forming lipid in a water-miscible organic solvent, preferably at a high concentration, and an aqueous medium, preferably in a small amount. One of the aspects of the present invention concerns a general gel hydration method of making liposomes, comprising the following steps:

(I) mixing at least one liposome-forming lipid, a water-miscible organic solvent and aqueous medium Y to form a gel or liquid containing gel particles without the creation of any gas/aqueous phase boundary; and thereafter

(II) (a) mixing the gel or liquid containing gel particles with aqueous medium Z1 to directly form the liposomes;
(b) (i) mixing the gel or liquid containing gel particles with aqueous medium Z1 to form a curd or curdy substance; and
   (ii) mixing the curd or curdy substance with aqueous medium Z2 to directly form the liposomes; or
(c) (i) cooling the gel or liquid containing gel particles to form a waxy substance; and
   (ii) mixing the waxy substance with aqueous medium Z1 to directly form the liposomes;

wherein aqueous media Y, Z1 and Z2 are the same or different.

In step (I) of the method, the gel or liquid containing gel particles is formed without the creation of any gas/aqueous phase boundary. The gel or liquid containing gel particles is formed by mixing the at least one liposome-forming lipid, the water-miscible organic solvent and aqueous medium Y without sonication or any other method (such as the application of high frequency energy to the mixture of the at least one liposome-forming lipid, the water-miscible organic solvent and aqueous medium Y) of producing a gas/aqueous phase boundary. The “high frequency energy” is the energy having a frequency at least equal to the frequency of ultrasound.

In certain embodiments of the method of preparing liposomes of the present invention, if the gel or liquid containing gel particles contains at least one acidic phospholipid, the content of the at least one acidic phospholipid is about 20% to about 100%, about 30% to about 100%, about 40% to about 100%, about 50% to about 100%, about 60% to about 100%, about 70% to about 100% or about 80% to about 100% by weight of the lipid(s) in the gel or liquid containing gel particles.

In certain embodiments of the method of preparing liposomes of the present invention, a phospholipid content of the gel or the liquid containing gel particles of step (I) is not 15% to 30% by weight of the gel.

In certain embodiments of the method of preparing liposomes of the present invention, a phospholipid content of the gel or the liquid containing gel particles of step (I) is not 15% to 30% by weight of the gel or the liquid containing gel particles and the content of the water-miscible organic solvent in the gel or the liquid containing gel particles is not 14% to 20% by weight of the gel or the liquid containing gel particles.

In certain embodiments of the method of preparing liposomes of the present invention, at least one charged lipid is added in step (I) to form the gel or the liquid containing gel particles. The at least one charged lipid and the at least one liposome-forming lipid are the same or different. If the at least one charged lipid is added in step (I) to form the gel or the liquid containing gel particles, the content of the at least one charged lipid in the gel or the liquid containing gel particles of step (I) can range from about 40% to about 100%, about 50% to about 100%, about 60% to about 100%, about 70% to about 100% or about 80% to about 100% by weight of the lipid(s) in the gel or the liquid containing gel particles. One of the benefits of adding at least one charged lipid in forming the liposomes is that the liposomes formed would have a small size, i.e., a preferred mean diameter, weighted by number, of about 400 nm or less, about 300 nm or less, about 200 nm or less, or about 100 nm or less, without the requirement of any sonication to form the gel or liquid containing gel particles, or the requirement of any sonication or extrusion of the liposomes.

In certain embodiments of the method of preparing liposomes of the present invention, at least one charged lipid and at least one acidic phospholipid are included in the liposomes. The at least one charged lipid and at least one acidic phospholipid are added in step (I) to form the gel or the liquid containing gel particles. The at least one charged lipid, the at least one acidid phospholipid and the at least one liposome-forming lipid are the same or different. The contents of the at least one charged lipid and at least one acidic phospholipid in the gel or the liquid containing gel particles are as disclosed above.

In certain embodiments of the method of preparing liposomes of the present invention, the formation of the gel or the liquid containing gel particles in step (I) does not involve the use of any hydrating agent, which is defined as a compound having at least two ionizable groups, one of which ionizable groups is capable of forming an easily dissociative ionic salt, which salt can complex with the ionic functionality of the liposome-forming lipid. The hydrating agent inherently does not form liposomes in and of itself and the hydrating agent must also be physiologically acceptable.

Within the scope of the present invention is a method for preparing liposomes containing at least one biologically active substance encapsulated therein, said method comprising the following steps:

(I) (A) mixing at least one liposome-forming lipid, the at least one biologically active substance, a water-miscible organic solvent and aqueous medium Y to form a gel or liquid containing gel particles without creation of any gas/aqueous phase boundary; or
(B) mixing at least one liposome-forming lipid, a water-miscible organic solvent and aqueous medium Y to form a gel or liquid containing gel particles without creation of any gas/aqueous phase boundary; and thereafter

(II) (A) mixing the gel or liquid containing gel particles of step (I) (A) with aqueous medium Z1 to directly form the liposomes containing the at least one biologically active substance encapsulated in the liposomes;
(B) (i) mixing the gel or liquid containing gel particles of step (I)(A) with aqueous medium Z1 to form a curd or curdy substance; and
   (ii) mixing the curd or curdy substance with aqueous medium Z2 to directly form the liposomes containing the at least one biologically active substance encapsulated in the liposomes;
(C) (i) cooling the gel or liquid containing gel particles of step (I)(A) to form a waxy substance; and
   (ii) mixing the waxy substance with aqueous medium Z1 to directly form the liposomes;
(D) mixing the gel or liquid containing gel particles of step (I)(B) with aqueous medium Z1 and the at least one biologically active substance to directly form the liposomes containing the at least one biologically active substance encapsulated in the liposomes;
(E) (i) mixing the gel or liquid containing gel particles of step (I)(B) with aqueous medium Z1 and the at least one biologically active substance to form a curd or curdy substance; and
   (ii) mixing the curd or curdy substance with aqueous medium Z2 to directly form the liposomes containing the at least one biologically active substance encapsulated in the liposomes;
(F) (i) mixing the gel or liquid containing gel particles of step (I)(B) with aqueous medium Z1 to form a curd or curdy substance; and
   (ii) mixing the curd or curdy substance with aqueous medium Z2 and the at least one biologically active substance to directly form the liposomes containing the at least one biologically active substance encapsulated in the liposomes; or
(G) (i) cooling the gel or liquid containing gel particles of step (I)(B) to form a waxy substance; and
   (ii) mixing the waxy substance with aqueous medium Z1 and the at least one biologically active substance to directly form the liposomes;

wherein aqueous media Y, Z1 and Z2 are the same or different.

In step (I) of the method of preparing liposomes containing the at least one biologically active substance encapsulated therein of the invention, the gel or liquid containing gel particles is formed without the creation of a gas/aqueous phase boundary. The gel or liquid containing gel particles is formed by mixing the at least one liposome-forming lipid, the water-miscible organic solvent and aqueous medium Y, optionally with the at least one biologically active substance, without sonication or any other method (such as the application of high frequency energy to the mixture of the at least one liposome-forming lipid, the water-miscible organic solvent and aqueous medium Y, optionally with the at least one biologically active substance) of producing a gas/aqueous phase boundary.

In certain embodiments of the method of preparing the liposomes containing the at least one biologically active substance encapsulated therein of the present invention, step (I)(A) is performed by

(a) (i) dissolving at least one liposome-forming lipid and the at least one biologically active substance in the water-miscible organic solvent to form a mixture; and
(ii) mixing the mixture with aqueous medium Y to form the gel or liquid containing gel particles; or

(b) (i) dissolving at least one liposome-forming lipid in the water-miscible organic solvent to form an organic solution; and
(ii) dissolving the at least one biologically active substance in aqueous medium Y to form an aqueous solution; and
(iii) mixing the organic solution and aqueous solution to form the gel or liquid containing gel particles.

In certain embodiments of the method of preparing liposomes containing the at least one biologically active substance encapsulated therein of the present invention, if the gel or liquid containing gel particles contains at least one acidic phospholipid, the content of the at least one acidic phospholipid is about 20% to about 100%, about 30% to about 100%, about 40% to about 100%, about 50% to about 100%, about 60% to about 100%, about 70% to about 100% or about 80% to about 100% by weight of the lipid(s) in the gel or liquid containing gel particles.

In certain embodiments of the method of preparing the liposomes encapsulating the biologically active substance of the present invention, a phospholipid content of the gel or the liquid containing gel particles in step (I) is not 15 to 30% by weight of the gel or the liquid containing gel particles.

In certain embodiments of the method of preparing the liposomes encapsulating the biologically active substance of the present invention, a phospholipid content of the gel or the liquid containing gel particles in step (I) is not 15 to 30% by weight of the gel or the liquid containing gel particles and the content of the water-miscible organic solvent is not 14 to 20% by weight of the gel or the liquid containing gel particles.

In certain embodiments of the method of preparing liposomes containing the at least one biologically active substance encapsulated therein of the present invention, at least one charged lipid is added in step (I) to form the gel or the liquid containing gel particles. The at least one charged lipid and the at least one liposome-forming lipid are the same or different. If the at least one charged lipid is added in step (I) to form the gel or the liquid containing gel particles, the content of the at least one charged lipid in the gel or the liquid containing gel particles of step (I) can range from about 40% to about 100%, about 50% to about 100%, about 60% to about 100%, about 70% to about 100% or about 80% to about 100% by weight of the lipid(s) in the gel or the liquid containing gel particles. One of the benefits of adding at least one charged lipid in forming the liposomes containing the at least one biologically active substance encapsulated therein is that the liposomes formed would have a small size, i.e., a preferred mean diameter, weighted by number, of about 400 nm or less, about 300 nm or less, about 200 nm or less, or about 100 nm or less, without the requirement of any sonication to form the gel or liquid containing gel particles, or the requirement of any sonication or extrusion of the liposomes.

In certain embodiments of the method of preparing liposomes containing the at least one biologically active substance encapsulated therein of the present invention, at least one charged lipid and at least one acidic phospholipid are included in the liposomes. The at least one charged lipid and at least one acidic phospholipid are added in step (I) to form the gel or the liquid containing gel particles. The at least one charged lipid, the at least one acidid phospholipid and the at least one liposome-forming lipid are the same or different. The contents of the at least one charged lipid and at least one acidic phospholipid in the gel or the liquid containing gel particles are as disclosed above.

In certain embodiments of the method of preparing the liposomes encapsulating the biologically active substance of the present invention, the formation of the gel or the liquid containing gel particles in step (I) does not involve the use of any hydrating agent.

In certain embodiments of the method of preparing the liposomes encapsulating the biologically active substance, in step (II)(A) or (II)(B) the gel or liquid containing the gel particles are mixed with aqueous medium Z1 and the biologically active substance; or in step (II)(C)(ii) the curd or curdy substance is mixed with aqueous medium Z2 and the biologically active substance.

### BRIEF DESCRIPTION OF THE DRAWINGS

FIG. 1 shows, under a light microscope (magnification 400×), N—C12-DOPE/DOPC (in a 70/30 molar ratio, with a volume ratio of aqueous phase:ethanol of 2:1) liposomes prepared according to the method of the present invention before (top panel) and after (bottom panel) extrusion through a membrane filter having a 0.4 μm pore size.

FIG. 2 depicts the appearance of N—C12-DOPE/DOPC (70/30) liposomes prepared according to the method of the present invention under freeze-fracture electron microscopy.

FIG. 3 depicts the appearance of N—C12-DOPE/DOPC (70/30) liposomes prepared according to the method of the present invention under cryo electron microscopy.

FIG. 4 shows the encapsulation efficiencies and particle sizes of N—C12-DOPE/DOPC (70/30) liposomes containing DNA prepared according to the method of the present invention. Three particle sizes were given for the samples in the order of: mean particle diameter weighted by number, mean particle diameter weighted by light reflection intensity and mean particle diameter weighted by volume. The particle sizes were below 400 nm. Also shown were the final DNA concentration, lipid concentration and ratio of DNA to lipid in the liposomes.

FIG. 5 shows the results of fractionation of N—C12-DOPE/DOPC liposomes prepared according to the method of the present invention in a 5-20% sucrose gradient. The lipids were homogeneously distributed with no phase separation. The liposomes in the peak fractions had entrapment of 2.1+/−0.2 μl/μmol of lipids. The open squares, labeled “p/pc”, represented the phosphate to choline molar ratios, as determined by the respective assays, of the fractions separated by the sucrose gradient.

FIG. 6 is the phase diagram of a lipids-ethanol-aqueous buffer system, wherein the lipids were N—C12-DOPE/DOPC (70/30). The three axes of the ternary phase diagram show the individual weight fractions of the three components (lipids, ethanol or aqueous buffer) based on the sum of the weight of the three components. In the region above line a, the mixture was a clear liquid. In the region between line a and line b, the mixture existed as a cloudly liquid. In the region between line b and line c, the mixture was in a clear gel state. In the region between line c and line d, the mixture existed as a cloudy gel. In the region below line d, the mixture became liposomes with the appearance of a cloudy liquid. Therefore, in the phase diagram, the region above line b was the fluid zone and the region below line d was the liposome zone with the intermediate region (between line b and line d) being the gel zone. A study showed that the presence of a EGFP plasmid DNA did not alter the lipids/ethanol/aqueous medium ternary phase diagram.

FIG. 7 shows the light scattering of 100 μg/ml enhanced green fluorescence protein (hereinafter referred to as EGFP) plasmid DNA in ethanol-LSB solution with or without 200 mM sodium chloride, wherein “LSB” represented “low salt buffer.” In the presence of 200 mM sodium chloride, the DNA started to aggregate at 30% (wt/wt) ethanol, while without 200 mM sodium chloride, the DNA started to aggregate at 55% (wt/wt) ethanol.

FIG. 8 shows the transfection of OVCAR-3 cells with N—C12-DOPE/DOPC (70/30) liposomes (washed to remove unencapsulated DNA) prepared by the gel-hydration method of the present invention using ethanol as the water-miscible organic solvent, wherein the liposomes (washed to remove unencapsulated DNA) contained EGFP plasmid DNA encapsulated therein. After incubation of the OVCAR-3 cells with the liposomes, the transfection activity was determined based on the expression of the EGFP plasmid DNA in the OVCAR-3 cells. The transfection activity did not require any plasmid DNA condensing agent or any extrusion, which was a liposome size reduction process.

FIG. 9 shows the transfection of OVCAR-3 cells with N—C12-DOPE/DOPC (70/30) liposomes (washed to remove unencapsulated DNA) prepared by the gel-hydration method of the present invention using ethanol as the water-miscible organic solvent, wherein the liposomes (washed to remove unencapsulated DNA) contained luciferase plasmid DNA encapsulated therein. After incubation of the OVCAR-3 cells with the liposomes, the transfection activity was determined based on the expression of the luciferase gene in the plasmid DNA in the OVCAR-3 cells. The liposomes could transfect the OVCAR-3 cells in the presence of 10% serum (FBS stands for fetal bovine serum) with or without targeting via transferrin.

FIG. 10 shows the transfection of OVCAR-3 cells with N—C12-DOPE/DOPC (70/30) liposomes prepared by the gel-hydration method of the present invention using ethanol as the water-miscible organic solvent, wherein the liposomes contained luciferase plasmid DNA encapsulated therein. After incubation of the OVCAR-3 cells with the liposomes at various concentrations of CaCl₂ and MgCl₂, the transfection activity was determined based on the expression of the luciferase gene in the plasmid DNA in the OVCAR-3 cells. The liposomes could transfect the OVCAR-3 cells at physiological Ca²⁺ and Mg²⁺ concentrations, i.e., about 1.2 mM Ca²⁺ and 0.8 mM Mg²⁺.

FIG. 11 shows the transferrin mediated binding of N—C12-DOPE/DOPC (70/30) liposomes prepared by the gel-hydration method of the present invention using ethanol as the water-miscible organic solvent (see Example 13). The binding experiment was conducted in the presence of 10% FBS.

FIG. 12 shows the transferrin mediated transfection of N—C12-DOPE/DOPC (70/30) liposomes prepared by the gel-hydration method of the present invention using ethanol as the water-miscible organic solvent, wherein the liposomes contained PGL-3 plasmid DNA encapsulated therein. The experiment was conducted in the presence of 10% FBS.

FIG. 13 shows the transfection activity of liposomes prepared with pure DOPC, DOPC/N—C12-DOPE (8:2 molar ratio), DOPC/N—C12-DOPE (6:4 molar ratio), DOPC/N—C12-DOPE (4:6 molar ratio), DOPC/N—C12-DOPE (2:8 molar ratio) or pure N—C12-DOPE using the gel hydration method of the present invention in OVCAR-3 cells in culture. After incubation of the cells with the liposomes, the expression of the EGFP gene in the cells was determined by measuring the intensity of green fluorescence.

FIG. 14 shows the encapsulation efficiencies, for dextran fluorophores, of N—C12-DOPE/DOPC (70/30) liposomes prepared using the gel hydration method of the present invention or using a process for making stable plurilamellar vesicles (SPLV). The N—C12-DOPE/DOPC liposomes prepared according to the gel-hydration method of the present invention had a much higher encapsulation efficiency than the N—C12-DOPE/DOPC liposomes prepared using the SPLV process.

FIG. 15 shows the captured volume, particle sizes and lamellarity of liposomes prepared according to the gel hydration method of the present invention, wherein the lamellarity was expressed as percent of lipid on the outer surface of the liposome.

FIG. 16 shows the ternary phase diagram of a lipid/water-miscible organic solvent/aqueous medium system, wherein the lipid was POPC, the water-miscible organic solvent was ethanol and the aqueous medium was a 100 mM Tris buffer. Varying amounts of the lipid were dissolved in ethanol to form a lipid solution. Different amounts of the 100 mM Tris buffer were mixed with the lipid solution until a gel was formed. The boundary between the solution zone and the gel zone was as indicated by the open circles and dotted line in the ternary phase diagram. Additional amounts of the 100 mM Tris buffer were added to the gel with mixing to form liposomes. The boundary between the gel zone and the liposome zone was as indicated by the open circles and dotted line in the ternary phase diagram. The solution zone, gel zone and the liposome zone were as labeled. In six different preparations (represented by six different symbols: stars, triangles, pentagons, inverted triangles, circles and squares), the 100 mM Tris buffer was added in small increments as shown by the individual symbols.

FIG. 17 is the ternary phase diagram of a lipid/water-miscible organic solvent/aqueous medium system, wherein the lipids were POPC and POPG in a 95:5 molar ratio, the water-miscible organic solvent was ethanol and the aqueous medium was a 100 mM Tris buffer. The boundary between the solution zone and the gel zone was as indicated by the open circles and the dotted line in the ternary phase diagram. The boundary between the gel zone and the liposome zone was as indicated by the open circles and the dotted line in the ternary phase diagram. In four different preparations (represented by four different symbols: diamonds, triangles, circles and squares), the 100 mM Tris buffer was added in small increments as shown by the individual symbols.

FIG. 18 is the ternary phase diagram of a lipid/water-miscible organic solvent/aqueous medium system, wherein the lipids were POPC and POPG in a 9:1 molar ratio, the water-miscible organic solvent was ethanol and the aqueous medium was a 100 mM Tris buffer. The boundary between the lipid solution zone and the gel zone was as indicated by the dashed line in the ternary phase diagram. Additional amounts of the 100 mM Tris buffer were added to the gel with mixing to form liposomes. The boundary between the gel zone and the liposome zone was as indicated by the dotted line in the ternary phase diagram. In three different preparations (represented by three different symbols: stars, circles and squares), the 100 mM Tris buffer was added in small increments as shown by the individual symbols.

### DETAILED DESCRIPTION OF THE INVENTION

The methods of preparing liposomes of the present invention involve hydration of a mixture of at least one liposome-forming lipid and a water-miscible organic solvent in the form of a gel or a liquid containing gel particles. In the mixture of the at least one liposome-forming lipid and the water-miscible organic solvent, the lipid is typically dissolved in the water-miscible organic solvent, preferably at a high concentration. The mixture is mixed with, typically a small amount of, an aqueous medium to form the gel or the liquid containing gel particles. Hydration of the gel leads to formation of liposomes without any additional manipulation, such as evaporation or sonication, normally required in prior art methods. Depending on the liposome-forming lipid used, in the methods of the present invention, upon hydration the gel or gel particles may go through a curd or curdy stage before forming liposomes, but no additional manipulation, such as evaporation or sonication, is required other than hydration of the gel or the gel particles in the liquid followed by hydration of a curd or curdy substance formed from the hydration of the gel or gel particles. For instance, when certain saturated liposome-forming lipids are used in the methods, the gel or gel particles go through the curd or curdy stage upon hydration before liposome formation. Alternatively, the gel or the liquid containing gel particles can be cooled to obtain a waxy substance, which upon hydration directly forms the liposomes without any further manipulation, such as sonication or evaporation, required.

In the method for preparing liposomes containing the biologically active substance encapsulated therein of the present invention, the gel or the gel particles in the liquid containing gel particles can be clear when first formed and turns cloudy upon further hydration. In one of the embodiments of the method for preparing liposomes containing the biologically active substance encapsulated therein, steps (I) and (II) are conducted by

(I) (A) (i) mixing the at least one liposome-forming lipid, the at least one biologically active substance, the water-miscible organic solvent and aqueous medium Y to form a clear gel or a liquid containing clear gel particles; and
(ii) mixing the clear gel or liquid containing clear gel particles with additional aqueous medium Y to form a cloudy gel or a liquid containing cloudy gel particles; or

(B) (i) mixing the at least one liposome-forming lipid, the water-miscible organic solvent and aqueous medium Y to form a clear gel or a liquid containing clear gel particles; and
(ii) mixing the clear gel or the liquid containing clear gel particles with additional aqueous medium Y to form a cloudy gel or a liquid containing cloudy gel particles; and thereafter

(II) (A) mixing the cloudy gel or liquid containing cloudy gel particles of step (I)(A) with aqueous medium Z1 to directly form the liposomes containing the at least one biologically active substance encapsulated in the liposomes; or
(B) (i) mixing the cloudy gel or liquid containing cloudy gel particles of step (I)(A) with aqueous medium Z1 to form a curd or curdy substance; and
   (ii) mixing the curd or curdy substance with aqueous medium Z2 to directly form the liposomes containing the at least one biologically active substance encapsulated in the liposomes;
(C) (i) cooling the cloudy gel or liquid containing cloudy gel particles of step (I)(A) to form a waxy substance; and
   (ii) mixing the waxy substance with aqueous medium Z1 to directly form the liposomes;
(D) mixing the cloudy gel or liquid containing cloudy gel particles of step (I)(B) with aqueous medium Z1 and the at least one biologically active substance to directly form the liposomes containing the at least one biologically active substance encapsulated in the liposomes;
(E) (i) mixing the cloudy gel or liquid containing cloudy gel particles of step (I)(B) with aqueous medium Z1 and the at least one biologically active substance to form a curd or curdy substance; and
   (ii) mixing the curd or curdy substance with aqueous medium Z2 to directly form the liposomes containing the at least one biologically active substance encapsulated in the liposomes;
(F) (i) mixing the cloudy gel or liquid containing cloudy gel particles of step (I)(B) with aqueous medium Z1 to form a curd or curdy substance; and
   (ii) mixing the curd or curdy substance with aqueous medium Z2 and the at least one biologically active substance to directly form the liposomes containing the at least one biologically active substance encapsulated in the liposomes; or
(G) (i) cooling the cloudy gel or liquid containing cloudy gel particles of step (I)(B) to form a waxy substance; and
   (ii) mixing the waxy substance with aqueous medium Z1 and the at least one biologically active substance to directly form the liposomes;

In one of the embodiments of the method of preparing liposomes of the present invention, after step (II) the liposomes are washed with an aqueous medium by centrifugation, gel filtration or dialysis.

Within the scope of the present invention is a method for preparing liposomes starting in the gel zone. This method for preparing liposomes comprises the following steps:

(i) providing a gel or a liquid containing gel particles comprising at least one liposome-forming lipid, a water-miscible organic solvent and aqueous medium Y; and thereafter

(ii) (a) mixing the gel or the liquid containing gel particles with aqueous medium Z to directly form the liposomes;
(b) (aa) mixing the gel or the liquid containing gel particles with aqueous medium Z to form a curd or curdy substance; and
   (bb) mixing the curd or curdy substance with additional aqueous medium Z to directly form the liposomes, or
(c) (aa) cooling the gel or the liquid containing gel particles to form a waxy substance; and
   (bb) mixing the waxy substance with aqueous medium Z to directly form the liposomes,

wherein aqueous media Y and Z are the same or different.

In certain embodiments of this method of preparing liposomes starting in the gel zone, a phospholipid content of the gel or the liquid containing gel particles in step (i) is not 15 to 30% by weight of the gel or the liquid. In certain embodiments of this method of preparing liposomes starting in the gel zone, a phospholipid content of the gel or the liquid containing gel particles in step (i) is not 15 to 30% by weight of the gel or the liquid containing gel particles and the content of the water-miscible organic solvent is not 14 to 20% by weight of the gel or the liquid containing gel particles.

In certain embodiments of this method of preparing liposomes starting in the gel zone of the present invention, at least one charged lipid is included in the gel or the liquid containing gel particles. The at least one charged lipid and the at least one liposome-forming lipid are the same or different. If the at least one charged lipid is included in the gel or the liquid containing gel particles, the content of the at least one charged lipid in the gel or the liquid containing gel particles used in step (i) can range from about 40% to about 100%, about 50% to about 100%, about 60% to about 100%, about 70% to about 100% or about 80% to about 100% by weight of the lipid(s) in the gel or the liquid containing gel particles. One of the benefits of adding at least one charged lipid in forming the liposomes is that the liposomes formed would have a small size, i.e., a preferred mean diameter, weighted by number, of about 400 nm or less, about 300 nm or less, about 200 nm or less, or about 100 nm or less, without the requirement of any sonication to form the gel or liquid containing gel particles, or the requirement of any sonication or extrusion of the liposomes.

The method of preparing liposomes containing the biologically active substance encapsulated therein of the present invention can also be modified to start in the gel zone. This method for preparing liposomes containing the at least one biologically active substance encapsulated therein comprises the following steps:

(i) (a) providing a gel or a liquid containing gel particles comprising at least one liposome-forming lipid, a water-miscible organic solvent, the least one biologically active substance and aqueous medium Y; or
(b) providing a gel or a liquid containing gel particles comprising at least one liposome-forming lipid, a water-miscible organic solvent, and aqueous medium Y; and thereafter

(ii) (a) mixing the gel or the liquid containing gel particles of step (i)(a) with aqueous medium Z to directly form the liposomes;
(b) (aa) mixing the gel or the liquid containing gel particles of step (i)(a) with aqueous medium Z to form a curd or curdy substance; and
   (bb) mixing the curd or curdy substance with additional aqueous medium Z to directly form the liposomes, or
(c) (aa) cooling the gel or the liquid containing gel particles of step (i)(a) to form a waxy substance; and
   (bb) mixing the waxy substance with aqueous medium Z to directly form the liposomes;
(d) mixing the gel or the liquid containing gel particles of step (i)(b) with the at least one biologically active substance and aqueous medium Z to directly form the liposomes;
(e) (aa) mixing the gel or the liquid containing gel particles of step (i) (b) with the at least one biologically active substance and aqueous medium Z to form a curd or curdy substance; and
   (bb) mixing the curd or curdy substance with additional aqueous medium Z to directly form the liposomes, or
(f) (aa) cooling the gel or the liquid containing gel particles of step (i)(b) to form a waxy substance; and
   (bb) mixing the waxy substance with the at least one biologically active substance and aqueous medium Z to directly form the liposomes,

wherein aqueous media Y and Z are the same or different.

In certain embodiments of this method of preparing liposomes containing the at least one biologically active substance encapsulated therein starting in the gel zone, at least one acidic phospholipid is included in the gel or the liquid containing gel particles. The at least one acidic phospholipid and the at least one liposome-forming lipid are the same or different. If the at least one acidic phospholipid is included in the gel or the liquid containing gel particles, the content of the at least one acidic phospholipid in the gel or the liquid containing gel particles used in step (i) can range from about 20% to about 100%, about 30% to about 100%, about 40% to about 100%, about 50% to about 100%, about 60% to about 100%, about 70% to about 100% or about 80% to about 100% by weight of the lipid(s) in the gel or the liquid containing gel particles.

In certain embodiments of this method of preparing liposomes containing the at least one biologically active substance encapsulated therein starting in the gel zone, at least one charged lipid is included in the gel or the liquid containing gel particles. The at least one charged lipid and the at least one liposome-forming lipid are the same or different. If the at least one charged lipid is included in the gel or the liquid containing gel particles, the content of the at least one charged lipid in the gel or the liquid containing gel particles used in step (i) can range from about 40% to about 100%, about 50% to about 100%, about 60% to about 100%, about 70% to about 100% or about 80% to about 100% by weight of the lipid(s) in the gel or the liquid containing gel particles. One of the benefits of adding at least one charged lipid in forming the liposomes containing the at least one biologically active substance encapsulated therein is that the liposomes formed would have a small size, i.e., a preferred mean diameter, weighted by number, of about 400 nm or less, about 300 nm or less, about 200 m or less, or about 100 nm or less, without the requirement of any sonication to form the gel or liquid containing gel particles, or the requirement of any sonication or extrusion of the liposomes.

In certain embodiments of this method of preparing liposomes containing the at least one biologically active substance encapsulated therein starting in the gel zone, at least one charged lipid and at least one acidic phospholipid are included in the gel or the liquid containing gel particles. The at least one charged lipid, the at least one acidid phospholipid and the at least one liposome-forming lipid are the same or different. The contents of the at least one charged lipid and at least one acidic phospholipid in the gel or the liquid containing gel particles are as disclosed above.

In certain embodiments of this method of preparing liposomes containing the biologically active substance encapsulated therein starting in the gel zone, a phospholipid content of the gel or the liquid containing gel particles in step (i) is not 15 to 30% by weight of the gel or the liquid. In certain embodiments of this method of preparing liposomes starting in the gel zone, a phospholipid content of the gel or the liquid containing gel particles in step (i) is not 15 to 30% by weight of the gel or the liquid containing gel particles and the content of the water-miscible organic solvent is not 14 to 20% by weight of the gel or the liquid containing gel particles.

In certain embodiments of the general gel hydration method and the method of preparing the liposomes containing the at least one biologically active substance encapsulated therein of the present invention, step (I) does not involve the use of any hydrating agent as defined above for the formation of the gel or the liquid containing gel particles. Preferably, the at least two ionizable groups of the hydrating agent are of opposite charge. Examples of the hydrating agent are arginine, homoarginine, γ-aminobutyric acid, glutamic acid, aspartic acid and similar amino acids.

In certain embodiments of the general gel hydration method starting in the gel zone and the method of preparing the liposomes containing the at least one biologically active substance encapsulated therein starting in the gel zone, the gel or the liquid containing gel particles contain no hydrating agent as defined above.

In the method of preparing liposomes by the general gel hydration method of the present invention or in the method of preparing the liposomes containing the at least one biologically active substance encapsulated therein of the present invention, “to directly form the liposomes” means that the liposomes are formed without requiring any additional procedure or manipulation, such as evaporation or sonication, other than going through a potential intermediate stage of formation of a curd or curdy substance if certain liposome-forming lipids are used or going through a waxy stage if the gel or the liquid containing gel particles are cooled. For instance, in the method of preparing the liposomes encapsulating the biologically active substance, mixing the gel or the liquid containing gel particles with aqueous medium Z1 in step (II)(A) leads directly to the formation of the liposomes having the biologically active substance entrapped without the requirement of any additional procedure or manipulation, such as evaporation or sonication.

The method of preparing liposomes of the invention can be used to encapsulate at least one biologically active substance in the liposomes. The at least one biologically active substance to be encapsulated can be either, if it is hydrophobic, co-dissolved with the at least one liposome-forming lipid in the water-miscible organic solvent or, if it is hydrophilic, dissolved in an aqueous medium, preferably at a high concentration. If the biologically active substance is co-dissolved with the lipid in the water-miscible organic solvent, an aqueous medium can be added at an appropriate volume ratio to create a gel. If the biologically active substance is dissolved in an aqueous medium to form an aqueous solution, the aqueous solution can be added to the mixture of the at least one liposome-forming lipid and the water-miscible organic solvent at an appropriate volume ratio to create a gel or a liquid containing gel particles.

In the general gel hydration method of the present invention for preparing liposomes, or in the method of preparing the liposomes containing the at least one biologically active substance encapsulated therein, the aqueous medium Y, aqueous medium Z1 and/or aqueous medium Z2 is preferably an aqueous buffer. Examples of the aqueous buffer include citrate buffer, Tris buffer, phosphate buffer and a buffer containing sucrose or dextrose.

In the methods of preparing the liposomes or the liposomes containing the at least one biologically active substance encapsulated therein of the present invention, the gel or the liquid containing gel particles and aqueous medium Z1 are mixed by either adding aqueous medium Z1 to the gel or the liquid containing gel particles, or adding or infusing the gel or the liquid containing gel particles into aqueous medium Z1.

The “liposome-forming lipid” is any lipid that is capable of forming liposomes. Typically, the “liposome-forming lipid” is a lipid that can form lipid bilayers. Examples of the liposome-forming lipid include phospholipids, glycolipids and sphingolipids. The phospholipids that are liposome-forming include phosphatidylcholine, phosphatidylserine, phosphatidylinositol, phosphatidylglycerol, diphosphatidylglycerol and N-acyl phospatidylethanolamine. Examples of the liposome-forming phospholipid include phospholipids selected from the group consisting of dioleoyl phosphatidylcholine, dipalmitoyl phosphatidylcholine, distearoyl phosphatidylcholine, dimyristoyl phosphatidylcholine, 1-palmitoyl-2-oleoyl-sn-glycero-3-phosphocholine, 1-oleoyl-2-palmitoyl-sn-glycero-3-phosphocholine, 1,2-dioleoyl-sn-glycero-3-[phospho-rac-(1-glycerol)], 1,2-dipalmitoyl-sn-glycero-3-[phospho-rac-(1-glycerol)], 1,2-distearoyl-sn-glycero-3-[phospho-rac-(1-glycerol)], 1,2-dimyristoyl-sn-glycero-3-[phospho-rac-(1-glycerol)], 1-palmitoyl-2-oleoyl-sn-glycero-3-[phospho-rac-(1-glycerol)], 1-oleoyl-2-palmitoyl-sn-glycero-3-[phospho-rac-(1-glycerol)], N-decanoyl phosphatidylethanolamine, N-dodecanoyl phosphatidylethanolamine and N-tetradecanoyl phosphatidylethanolamine.

Preferably, the at least one liposome-forming lipid is phosphatidylcholine, e.g., dioleoyl phosphatidylcholine, dipalmitoyl phosphatidylcholine, distearoyl phosphatidylcholine, dimyristoyl phosphatidylcholine, 1-palmitoyl-2-oleoyl-sn-glycero-3-phosphocholine and 2-palmitoyl-1-oleoyl-sn-glycero-3-phosphocholine, or N-acyl phosphatidylethanolamine, e.g., 1,2-dioleoyl-sn-glycero-N-decanoyl-3-phosphoethanolamine, 1,2-dioleoyl-sn-glycero-N-dodecanoyl-3-phosphoethanolamine, 1,2-dioleoyl-sn-glycero-N-tetradecanoyl-3-phosphoethanolamine, 1,2-dipalmitoyl-sn-glycero-N-decanoyl-3-phosphoethanolamine, 1,2-dipalmitoyl-sn-glycero-N-dodecanoyl-3-phosphoethanolamine, 1,2-dipalmitoyl-sn-glycero-N-tetradecanoyl-3-phosphoethanolamine, 1-oleoyl-2-palmitoyl-sn-glycero-N-decanoyl-3-phosphoethanolamine, 1-oleoyl-2-palmitoyl-sn-glycero-N-dodecanoyl-3-phosphoethanolamine, 1-oleoyl-2-palmitoyl-sn-glycero-N-tetradecanoyl-3-phosphoethanolamine, 1-palmitoyl-2-oleoyl-sn-glycero-N-decanoyl-3-phosphoethanolamine, 1-palmitoyl-2-oleoyl-sn-glycero-N-dodecanoyl-3-phosphoethanolamine, and 1-palmitoyl-2-oleoyl-sn-glycero-N-tetradecanoyl-3-phosphoethanolamine.

Certain embodiments of the preparatory methods of the present invention use one, or a combination (at any ratio), of the following lipids: phosphatidylcholines, phosphatidylglycerols, phosphatidylserines, phosphatidylethanolamines, phosphatidylinositols, headgroup modified phospholipids, headgroup modified phosphatidylethanolamines, lyso-phospholipids, phosphocholines (ether linked lipids), phosphoglycerols (ether linked lipids), phosphoserines (ether linked lipids), phosphoethanolamines (ether linked lipids), sphingomyelins, sterols, such as cholesterol hemisuccinate, tocopherol hemisuccinate, ceramides, cationic lipids, monoacyl glycerol, diacyl glycerol, triacyl glycerol, fatty acids, fatty acid methyl esters, single-chain nonionic lipids, glycolipids, lipid-peptide conjugates and lipid-polymer conjugates. These lipids may or may not be liposome-forming. The lipid or a combination thereof are included in the gel or the liquid containing gel particles, added to the gel or the liquid containing gel particles or added during the hydration of the gel or the liquid containing gel particles in the methods of preparing the liposomes or the liposomes having the at least one biologically active substance encapsulated therein of the present invention. If the lipid(s) is liposome-forming, the lipid(s) can be added alone in step (I) or along with at least one other liposome-forming lipid in step (I) to form the gel or the liquid containing gel particles is formed. However, in certain embodiments of the methods of preparing the liposomes, with or without the at least one biologically active substance encapsulated therein, of the present invention, no phosphatidylcholine is used.

In certain embodiments of the methods of preparing liposomes of the present invention, at least one charged lipid is added in preparing the gel or the liquid containing gel particles. The at least one charged lipid and the at least one liposome-forming lipid are the same or different. The at least one charged lipid is the same or different from the at least one liposome-forming lipid. The “charged lipid” is a lipid having a net negative or positive charge in the molecule. Examples of the charged lipid include N-acyl phosphatidylethanolamine, phosphatidylserine, phosphatidylinositol, phosphatidylglycerol, diphosphatidylglycerol (i.e. cardiolipin) and phosphatidic acid.

The liposomes prepared by any of the methods of the present invention preferably comprises at least one fusogenic lipid. The gel in the methods of the present invention preferably further comprises the at least one fusogenic lipid. Preferably, the at least one liposome-forming lipid is also a fusogenic lipid. For instance, when the at least one liposome-forming lipid is a N-acyl phosphatidylethanolamine, the N-acyl phosphatidylethanolamine is liposome-forming and also increases the fusogenicity of the liposomes (see Meers et al, U.S. Pat. No. 6,120,797, the disclosure of which is herein incorporated by reference). N-acyl phosphatidylethanolamine that can be used include 1,2-dioleoyl-sn-glycero-N-decanoyl-3-phosphoethanolamine, 1,2-dioleoyl-sn-glycero-N-dodecanoyl-3-phosphoethanolamine, 1,2-dioleoyl-sn-glycero-N-tetradecanoyl-3-phosphoethanolamine, 1,2-dipalmitoyl-sn-glycero-N-decanoyl-3-phosphoethanolamine, 1,2-dipalmitoyl-sn-glycero-N-dodecanoyl-3-phosphoethanolamine, 1,2-dipalimitoyl-sn-glycero-N-tetradecanoyl-3-phosphoethanolamine, 1-oleoyl-2-palmitoyl-sn-glycero-N-decanoyl-3-phosphoethanolamine, 1-oleoyl-2-palmitoyl-sn-glycero-N-dodecanoyl-3-phosphoethanolamine, 1-oleoyl-2-palmitoyl-sn-glycero-N-tetradecanoyl-3-phosphoethanolamine, 1-palmitoyl-2-oleoyl-sn-glycero-N-decanoyl-3-phosphoethanolamine, 1-palmitoyl-2-oleoyl-sn-glycero-N-dodecanoyl-3-phosphoethanolamine, and 1-palmitoyl-2-oleoyl-sn-glycero-N-tetradecanoyl-3-phosphoethanolamine. The fusogenicity-increasing N-acyl phosphatidylethanolamine is preferably N-dodecanoyl phosphatidylethanolamine and more preferably 1,2-dioleoyl-sn-glycero-N-dodecanoyl-3-phosphoethanolamine.

The gel in step (I) or step (i) of the methods of the present invention can further comprise a sterol. Preferably, the sterol is cholesterol.

In the methods of the present invention, the water-miscible organic solvent is an organic solvent that, when mixed with water, forms a homogeneous liquid, i.e., with one phase. The water-miscible organic solvent can be selected from the group consisting of acetaldehyde, acetone, acetonitrile, allyl alcohol, allylamine, 2-amino-1-butanol, 1-aminoethanol, 2-aminoethanol, 2-amino-2-ethyl-1,3-propanediol, 2-amino-2-methyl-1-propanol, 3-aminopentane, N-(3-aminopropyl)morpholine, benzylamine, bis(2-ethoxyethyl)ether, bis(2-hydroxyethyl)ether, bis(2-hydropropyl)ether, bis(2-methoxyethyl)ether, 2-bromoethanol, meso-2,3-butanediol, 2-(2-butoxyethoxy)-ethanol, butylamine, sec-butylamine, tert-butylamine, 4-butyrolacetone, 2-chloroethanol, 1-chloro-2-propanol, 2-cyanoethanol, 3-cyanopyridine, cyclohexylamine, diethylamine, diethylenetriamine, N,N-diethylformamide, 1,2-dihydroxy-4-methylbenzene, N,N-dimethylacetamide, N,N-dimethylformaide, 2,6-dimethylmorpholine, 1,4-dioxane, 1,3-dioxolane, dipentaerythritol, ethanol, 2,3-epoxy-1-propanol, 2-ethoxyethanol, 2-(2-ethoxyethoxy)-ethanol, 2-(2-ethoxyethoxy)-ethyl acetate, ethylamine, 2-(ethylamino)ethanol, ethylene glycol, ethylene oxide, ethylenimine, ethyl(−)-lactate, N-ethylmorpholine, ethyl-2-pyridine-carboxylate, formamide, furfuryl alcohol, furfurylamine, glutaric dialdehyde, glycerol, hexamethylphosphor-amide, 2,5-hexanedione, hydroxyacetone, 2-hydroxyethyl-hydrazine, N-(2-hydroxyethyl)-morpholine, 4-hydroxy-4-methyl-2-pentanone, 5-hydroxy-2-pentanone, 2-hydroxypropionitrile, 3-hydroxypropionitrile, 1-(2-hydroxy-1-propoxy)-2-propanol, isobutylamine, isopropylamine, 2-isopropylamino-ethanol, 2-mercaptoethanol, methanol, 3-methoxy-1-butanol, 2-methoxyethanol, 2-(2-methoxyethoxy)-ethanol, 1-methoxy-2-propanol, 2-(methylamino)-ethanol, 1-methylbutylamine, methylhydrazine, methyl hydroperoxide, 2-methylpyridine, 3-methylpyridine, 4-methylpyridine, N-methylpyrrolidine, N-methyl-2-pyrrolidinone, morpholine, nicotine, piperidine, 1,2-propanediol, 1,3-propanediol, 1-propanol, 2-propanol, propylamine, propyleneimine, 2-propyn-1-ol, pyridine, pyrimidine, pyrrolidine, 2-pyrrolidinone and quinoxaline.

Acetonitrile, C₁-C₃ alcohols and acetone are preferred examples of the water-miscible organic solvent. The C₁-C₃ alcohols are preferably methanol, ethanol, 1-propanol, 2-propanol, ethylene glycol and propylene glycol, and more preferably ethanol, 1-propanol or 2-propanol, with ethanol being the most preferred. One of the advantages of the method of the present invention is that an organic solvent, such as ethanol or acetone, of relatively low toxicity can be used. With a water-miscible organic solvent of relatively low toxicity, the liposomes prepared according to the method of the present invention would not be expected to pose any significant toxic threat even when the liposomes contain a residual amount of the water-miscible organic solvent.

In the method of preparing liposomes of the present invention or the method of preparing liposomes containing the at least one biologically active substance encapsulated therein of the present invention, the amount of the at least one liposome-forming lipid in the gel or the liquid containing gel particles of step (I) or step (i) can range from about 1% by weight of the gel or the liquid containing gel particles to the hydration limit of the at least one liposome-forming lipid in water. The “hydration limit” of a liposome-forming lipid is the maximum amount of the liposome-forming lipid in a given amount of water that would keep the liposome-forming lipid in a liposomal state. The amount of the at least one liposome-forming lipid in the gel or the liquid containing gel particles of step (I) or step (i) can have a lower limit of about 5%, about 10%, about 15%, about 20%, about 30%, about 40%, about 50%, about 60% or about 70% by weight of the gel or the liquid containing gel particles, and an upper limit of about 95% by weight of the gel or the liquid containing gel particles. The amount of the at least one liposome-forming lipid in the gel or the liquid containing gel particles of step (I) or step (i) can have a lower limit of about 5%, about 10%, about 15%, about 20%, about 30%, about 40%, about 50%, about 60% or about 70% by weight of the gel or the liquid containing gel particles, and an upper limit of about 90% by weight of the gel or the liquid containing gel particles. The amount of the at least one liposome-forming lipid in the gel or the liquid containing gel particles of step (I) or step (i) can have a lower limit of about 5%, about 10%, about 15%, about 20%, about 30%, about 40%, about 50%, about 60% or about 70% by weight of the gel or the liquid containing gel particles, and an upper limit of about 85% by weight of the gel or the liquid containing gel particles. The amount of the at least one liposome-forming lipid in the gel or the liquid containing gel particles of step (I) or step (i) can also be from about 5% to about 80%, about 10% to about 80%, about 15% to about 80%, about 20% to about 80%, about 30% to about 80%, about 40% to about 80%, about 50% to about 80%, about 60% to about 80%, about 70% to about 80%, about 10% to about 70%, about 20% to about 60%, or about 30% to about 50% by weight of the gel or the liquid containing gel particles. Alternatively, the amount of the at least one liposome-forming lipid in the gel or the liquid containing gel particles of step (I) or step (i) ranges from about 60% to about 90%, or is about 45%, by weight of gel or the liquid containing gel particles.

In step (II) or step (ii) of the general gel hydration method of preparing the liposomes or the method of preparing the liposomes containing the at least one biologically active substance encapsulated therein, aqueous medium Z1 is preferably mixed with the gel or the liquid containing gel particles in increments. Mixing in increments has the advantage of yielding a higher entrapment efficiency compared with mixing the entire amount of the aqeuous medium Z1 with the gel or the liquid containing gel particles in one step. The size of the increment can be up to about 1000%, up to about 500%, up to about 200%, up to about 100%, up to about 90%, up to about 80%, up to about 70%, up to about 60%, up to about 50%, up to about 40%, up to about 30%, up to about 20%, up to about 10%, up to about 5%, up to about 2%, up to about 1%, up to about 0.5%, up to about, 0.1%, up to about 0.05% or up to about 0.01% of the weight of the gel or the liquid containing gel particles before the gel or the liquid is mixed with any aqueous medium Z1. The size of the increment can also be from about 0.001% to about 10%, from about 0.001% to about 5%, from about 0.001% to about 1% or from about 0.001% to about 0.1% of the weight of the gel or the liquid containing gel particles before the gel or the liquid is mixed with any aqueous medium Z1.

FIGS. 6 and 16-18 show the phase diagrams of several lipid(s)/water-miscible organic solvent/aqueous medium systems used in the gel hydration method of the present invention, wherein the lipid(s) were N—C12-DOPE/DOPC (70/30), pure POPC, POPC/POPG (95:5) and POPC/POPG (9:1). Ethanol was the water-miscible organic solvent and Tris buffer was the aqueous medium. The three axes of the ternary phase diagrams show the individual weight fractions of the three components (lipids, ethanol or aqueous buffer). In the ternary phase diagram, the liquid or solution zone, the gel zone and the liposome zone are depicted. Similar ternary phase diagrams can be generated by a person skilled in the art without undue experimentation for other lipid(s)/water-miscible organic solvent/aqueous medium systems. The method of the present invention can, however, be practiced without the ternary phase diagrams. The ternary phase diagrams are merely used herein to show the general relationship between the fluid zone, gel zone and liposome zone for the lipid(s)/water-miscible organic solvent/aqueous medium systems used in the methods of the present invention.

Liposomes are useful as delivery vehicles of encapsulated substances. The method of the present invention can be used to encapsulate at least one biologically active substance in liposomes. The liposomes containing the at least one biologically active substance encapsulated therein prepared by the method of the present invention have the advantages of a high entrapment efficiency and a relatively homogeneous particle size. Due to the simplicity of the procedures, the method of preparing the liposomes of the present invention allows relatively rapid production of the liposomes at a low cost. The method of the present invention has the additional advantage of being easily controlled and modified, e.g., by selecting a batch or continuous operation, to fit the special requirements of different formulations.

The at least one biologically active substance encapsulated in the liposomes prepared by the method of the present invention includes a pharmaceutical agent, nucleic acid, protein, peptide, diagnostic agent, antigen and hapten, especially a protein or antigen structurally sensitive to dehydration (e.g., solvent exposure at an air to water interface). The “antigen” that can be encapsulated includes toxoids. The “protein or antigen structurally sensitive to dehydration” is a protein or antigenic substance that loses structural integrity upon an exposure to dehydration, e.g., in an air to water interface. Examples of the “protein or antigen structurally sensitive to dehydration” are certain toxoids, e.g., tetanus toxoids.

Examples of the pharmaceutical agent that can be encapsulated in the liposomes are anti-neoplastic agents, anti-microbial agents, anti-viral agents, antihypertensive agents, anti-inflammatory agents, bronchodilators, local anesthetics and immunosuppressants. Preferably, the pharmaceutical agent is selected from the group consisting of anti-neoplastic agents, e.g., doxorubicin, anti-bacterial agents and anti-fungal agents, e.g., amphotericin B. Since systemic delivery of hydrophobic pharmaceutical agents is usually a problem due to the poor water solubility of the agents, liposomes are especially useful as delivery vehicles for hydrophobic pharmaceutical agents because the liposomes contain a significant amount of lipids with which the hydrophobic pharmaceutical agents can associate. As a result, the at least one pharmaceutical agent to be encapsulated in the liposomes prepared by the method of the present invention can be hydrophobic. For instance, bioactive lipids are especially suited for encapsulation in the liposomes prepared by the method of the present invention.

The at least one biologically active substance that is encapsulated in the liposomes prepared by the method of the present invention can be a diagnostic agent. Examples of the diagnostic agents include dyes, radioactive diagnostic agents and antibodies.

The at least one biologically active substance can also be a protein, such as an antibody, proteinaceous antigen, enzyme, cytochrome C, cytokine, toxoid, toxin (e.g., tetanoid toxin) and transcription factor.

In some of the embodiments of the present invention, the at least one biologically active substance is a nucleic acid, including an oligonucleotide, RNA and DNA. The oligonucleotide that can be encapsulated as the biologically active substance by the liposomes prepared by the method of the present invention can be of about 5 to about 500 bases in size. Examples of RNA that can be encapsulated in the liposomes prepared according to the present invention are anti-sense RNA and RNA interference or RNAᵢ.

The DNA that can be encapsulated in the liposomes prepared according to the present invention includes a plasmid DNA. The plasmid DNA can be of up to 20 kb, up to 15 kb, up to 10 kb, from about 0.5 kb to about 20 kb, from about 1 kb to about 15 kb, from about 2 kb to about 10 kb or from about 3 kb to about 7 kb in size. Liposomes of the present invention containing the plasmid DNA are useful in gene therapy, transfection of eukaryotic cells and transformation of prokaryotic cells. It was discovered that the liposomes prepared by the method of the present invention containing a plasmid DNA encapsulated therein have a high transfection efficiency.

The liposomes of the present invention having at least one biologically active substance encapsulated therein can be administered to a subject in need of the biologically active substance via an oral or parenteral route (e.g., intravenous, intramuscular, intraperitoneal, subcutaneous and intrathecal routes) for therapeutic or diagnostic purposes. The dose of the liposomes to be administered is dependent on the biologically active substance involved, and can be adjusted by a person skilled in the art based on the health of the subject and the medical condition to be treated or diagnosed. For diagnostic purposes, some the liposomes of the present invention can be used in vitro.

Within the scope of the present invention is a method of preventing or treating a health disorder in a subject in need of the treatment or prevention, said method comprises administering the liposomes containing a biologically active substance encapsulated therein as prepared by one of the above methods in the subject, wherein the biologically active substance is a pharmaceutical agent, protein, peptide, antibody or nucleic acid.

Also within the scope of the present invention is a method of diagnosing a health disorder, said method comprises using the liposomes containing a biologically active substance encapsulated therein as prepared by one of the above methods in a diagnostic test by mixing the liposomes with cells or a biological material obtained from a subject in need of the diagnosis, wherein the biologically active substance is a diagnostic agent, to deliver the diagnostic agent to the cells or biological material.

Additionally, the present invention encompasses a method of transfecting cells with a DNA, said method comprises using the liposomes containing a biologically active substance encapsulated therein, wherein the biologically active substance is the DNA, as prepared by one of the above methods by mixing the liposomes with the cells with optional incubation. The DNA preferably is a plasmid DNA. The plasmid DNA preferably contains a gene of interest for the transfection. Therefore, the liposomes prepared by the method of the present invention containing the plasmid DNA are useful in gene therapy, transfection of eukaryotic cells and transformation of prokaryotic cells. An aspect of the invention is a method for transfecting cells, preferably mammalian cells such as human cells, said method comprising contacting the cells in vivo or in vitro with the liposomes prepared containing the plasmid DNA encapsulated therein as prepared by the method of the present invention, wherein the plasmid DNA preferably contains a gene of interest. The transfection method is also useful in a method for gene therapy comprising contacting target cells of a subject in need of the gene therapy with the liposomes containing the plasmid DNA encapsulated therein, in vitro (e.g., via incubation) or in vivo (e.g., via administration of the liposomes into the subject), wherein the plasmid DNA contains a gene having the desired therapeutic effect on the subject. Within the scope of the invention is a method of transforming prokaryotic cells comprising contacting (e.g., via incubation) the prokaryotic cells with the liposomes containing a plasmid DNA encapsulated therein as prepared by the method of the present invention to obtain transformation of the prokaryotic cells.

The liposomes containing the biologically active substance encapsulated therein prepared by the method of the present invention can further comprise a targeting agent to facilitate the delivery of the biologically active substance to a proper target in a biological system. Examples of the targeting agent include antibodies, a molecule containing biotin, a molecule containing streptavidin, or a molecule containing a folate or transferrin molecule.

Some aspects of the present invention are shown in the following working examples. However, the scope of the present invention is not to be limited by the working examples. A person skilled in the art can practice the present invention as recited in the claims beyond the breadth of the working examples. The working examples are included for illustration purposes only.

The names of certain chemicals used in the working examples were abbreviated as shown below: 1,2-dioleoyl-sn-glycero-3-phosphoethanolamine-N-dodecanoyl (N—C12-DOPE); 1,2-dioleoyl-sn-glycero-3-phosphocholine (DOPC); 1-palmitoyl-2-oleoyl-sn-glycero-3-phosphocholine (POPC); 1-palmitoyl-2-oleoyl-sn-glycero-3-[phospho-rac-(1-glycerol)](POPG); 1,2-distearoyl-sn-glycero-3-phosphocholine (DSPC); 1,2-distearoyl-sn-glycero-3-[phospho-rac-(1-glycerol)] (DSPG) and enhanced green fluorescence protein plasmid DNA (EGFP plasmid DNA).

### EXAMPLE 1

### N—C12-DOPE/DOPC Liposome Preparation by Ethanol Gel Hydration

Typically, 36.7 mg of N—C12-DOPE and 14.2 mg of DOPC were co-dissolved in 100 μl ethanol. A volume of 100-200 μl of an aqueous solution containing a biological active substance was injected into the lipid ethanol solution under intense mixing. Then 1.8 ml of a hydration buffer (300 mM sucrose, 10 mM Tris, 1 mM NaCl, pH 7.0) was slowly added to the sample to form a suspension of liposomes. Any unencapsulated material was removed by washing (one wash consisted of (1) sedimenting the liposomes in an aqueous phase, (2) replacing the supernatant with fresh aqueous phase, and (3) resuspending the pellet) the liposomes three times via 10,000 g centrifugation.

If the biologically active substance to be encapsulated was a EGFP plasmid DNA or PGL-3 plasmid, and the liposome-forming lipid to be used was a mixture of N—C12-DOPE/DOPC (in a molar ratio of 70/30), generally the following procedure could be used to prepare the liposomes with gel hydration. The lipid mixture, N—C12-DOPE/DOPC (in a molar ratio of 70/30), was dissolved in ethanol at a concentration of about 600 mM. The plasmid DNA was added in an aqueous solution at a concentration of about 1 to 4 mg/ml to the lipid ethanol solution to form a clear gel. The gel was hydrated by adding an aqueous buffer (10 mM Tris, 1 mM sodium chloride, 300 mM sucrose, pH 7.0) under intense mixing. The gel turned cloudy and finally collapsed after additional aqueous solution was added. The so formed liposome suspension was washed by centrifugation to remove any free plasmid DNA.

### EXAMPLE 2

### Light Microscopy of N—C12-DOPE/DOPC Liposomes Prepared by Ethanol Gel Hydration

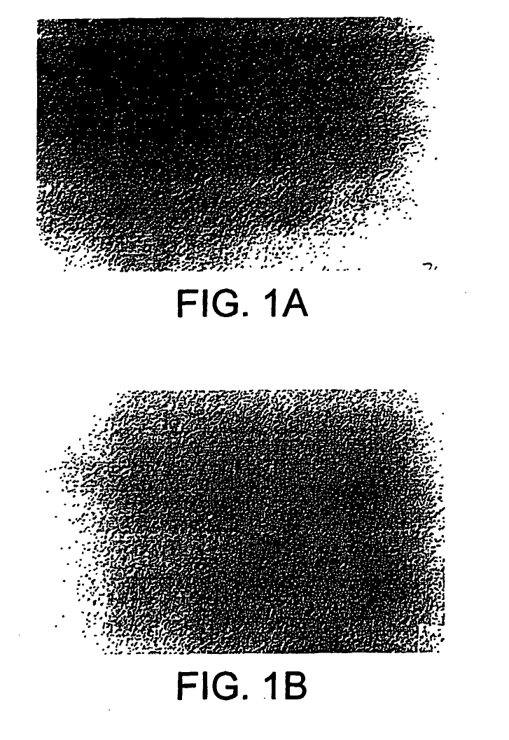

N—C12-DOPE/DOPC liposomes (70:30, molar ratio) were prepared by the gel hydration process (as set forth in Example 1) using 36.7 mg of N—C12-DOPE, 14.2 mg of DOPC and 400 μg of EGFP plasmid DNA. Light micrographs (Olympus BH-2, New York/New Jersey Scientific) of these liposomes before and after five passes of extrusion through a membrane filter with 400 nm pore size were taken at a magnification of 400× (see FIG. 1, top and bottom panels).

### EXAMPLE 3

### Freeze Fracture Electron Microscopy of N—C12-DOPE/DOPC Liposomes Prepared by Ethanol Gel Hydration

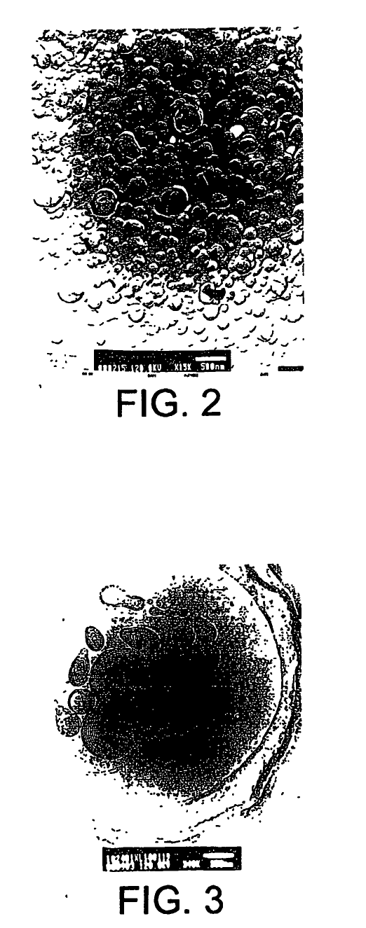

N—C12-DOPE/DOPC liposomes (70:30, molar ratio) were prepared by the gel hydration process (as set forth in Example 1) using 36.7 mg of N—C12-DOPE, 14.2 mg of DOPC and 400 μg PGL-3 plasmid DNA (a commercially available plasmid DNA containing luciferase as a reporter gene). Freeze fracture electron replicas were made and observed at magnifications of about 43,000× (see FIG. 2).

### EXAMPLE 4

### Cryo Electron Microscopy of N—C12-DOPE/DOPC Liposomes Prepared by Ethanol Gel Hydration

N—C12-DOPE/DOPC liposomes (70:30, molar ratio) were prepared by the gel hydration process (as set forth in Example 1) using 36.7 mg of N—C12-DOPE, 14.2 mg of DOPC and 400 μg of EGFP plasmid DNA. Liposomes samples were placed on Quantifoil^{ò} 2/2 grids, blotted with a filtering paper to form a uniform thin film of liquid 1-2 mm in thickness, and flush-frozen by plunging into liquid ethane. Frozen samples were transferred to a Gatan 910 cryo-holder and observed at a magnification of 30,000× at an accelerating voltage of 120 kV in a Jeol JEM-1200EX electron microscope (FIG. 3).

### EXAMPLE 5

### Particle Size Analysis

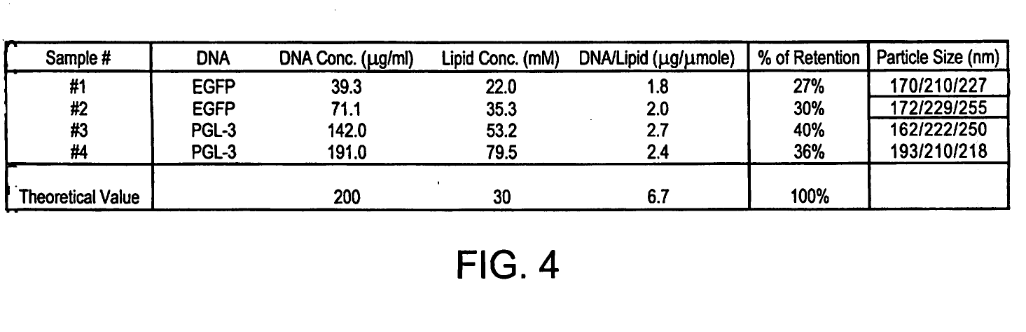

N—C12-DOPE/DOPC liposomes (70:30, molar ratio) were prepared by the gel hydration process (as set forth in Example 1) using 36.7 mg of N—C12-DOPE, 14.2 mg of DOPC and 400 μg PGL-3 or EGFP plasmid DNA. Their particle sizes were measure by a Submicron Particle Sizer (model 370), from NICOMP Particle Sizing Systems, Inc. Mean particle diameters (nm), as weighted by number, intensity or volume, were smaller than 400 nm (FIG. 4).

### EXAMPLE 6

### DNA to Lipid Ratio Measurement

N—C12-DOPE/DOPC liposomes (70:30, molar ratio) were prepared by the gel hydration process (as set forth in Example 1) using 36.7 mg of N—C12-DOPE, 14.2 mg of DOPC and 400 μg PGL-3 or EGFP plasmid DNA. The liposomes had DNA:lipid ratios of about 1-2 μg/μmole (FIG. 4), as determined by a phosphate assay and Picogreen assay (Shangguan et al., Gene Therapy, 769-783, 2000), respectively. The plasmid DNA was protected against DNase I digestion as described in Shangguan et al.

### EXAMPLE 7

### Sucrose Gradient Fractions of N—C12-DOPE/DOPC Liposomes Prepared by Ethanol Gel Hydration

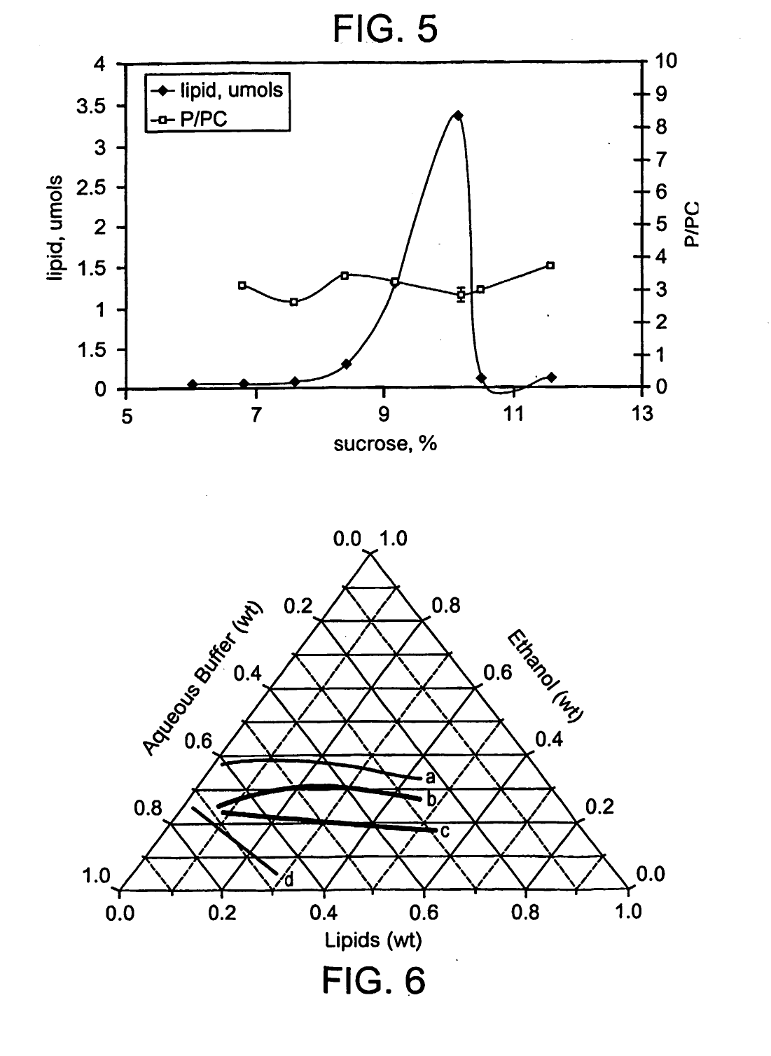

A 5-20% continuous sucrose gradient was obtained by mixing a 10 mM Tris buffer, pH 7, containing 140 mM NaCl, and a 10 mM Tris buffer, pH 7, containing 20% sucrose instead of NaCl. The liposomes were loaded on top of the gradient and centrifuged for 17 hours at 35,000 rpm. The centrifugation yielded a single band of liposomes centered at approximately 10% sucrose. The contents of the centrifuge tubes were fractionated starting from the bottom. The concentrations of the total phosholipids and DOPC were determined using phosphate and choline assays. In all fractions examined, the phosphate to choline ratios were nearly the same: 3±0.2 (see FIG. 5), which indicates compositional homogeneity of mixed lipid liposomes.

### EXAMPLE 8

### N—C12-DOPE/DOPC-Ethanol-Aqueous Phase Diagram

Different amounts of 560 mg of N—C12-DOPE/DOPC lipid mixtures (70:30, molar ratio) were dissolved in 38-190 mg ethanol to reach lipid concentrations of 3%, 14%, 18%. 25%, 31%, 40%, and 60% (wt/wt). A 5 mM HEPES buffer (pH 7.5) was added incrementally to the lipid solutions at increments of 20-25 mg under intense mixing. The total weight of added buffer was recorded each time when the mixtures underwent a phase change. Similarly, 25.5-60 mg of N—C12-DOPE/DOPC lipid mixtures (70:30, molar ratio) were suspended in 34-77 mg of a 5 mM HEPES buffer (pH 7.5) to reach lipid concentrations of 25%, 33%, 43%, and 60% (wt/wt). Ethanol was added incrementally to the lipid suspensions at increments of 15-30 mg under intense mixing. The total weight of added ethanol was recorded each time when the mixtures underwent a phase change. A ternary lipids-ethanol-aqueous phase diagram was constructed by connecting the critical points at which the mixture underwent any phase change (FIG. 6).

### EXAMPLE 9

### DNA Light Scattering in Ethanol Solutions

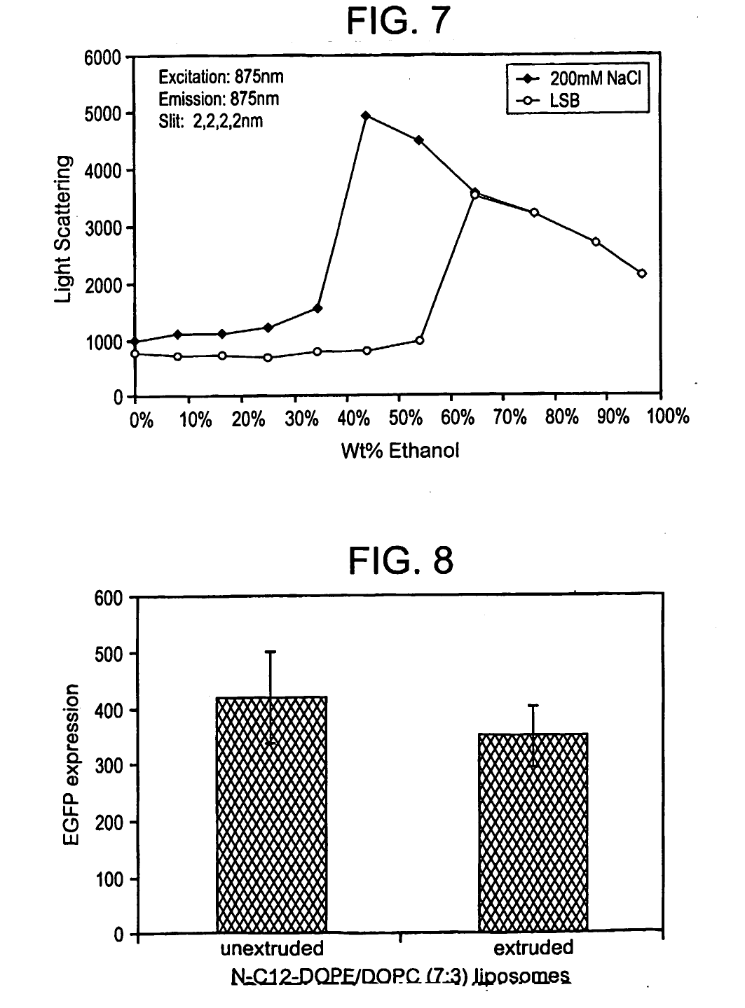

A volume of 85.7 μl of a EGFP plasmid DNA stock solution (3.5 mg EGFP plasmid DNA/ml) was added to each of 0-97% (wt/wt) ethanol solutions. In another experiment, the ethanol solution contained 200 mM NaCl. 90° light scattering of the EGFP plasmid DNA at 875 nm in different ethanol solutions was presented in FIG. 7. This experiment was conducted to determine the effect of ethanol on the plasmid DNA. The 200 mM NaCl solution was used to mimic the ionic strength in the gel containing N—C12-DOPE.

### EXAMPLE 10

### Transfection Activity of N—C12-DOPE/DOPC (70:30) Liposomes Made by the Gel Hydration Method (FIG. 8)

The N—C12-DOPE/DOPC (70:30) liposomes containing the EGFP plasmid DNA were made by the gel hydration method as set forth in Example 1. Half of the sample was extruded through a 400 nm filter five times before removal of unencapsulated DNA. For a transfection assay, OVCAR3 cells were plated in 96 well plates at 2×10⁵ cells/ml in 0.1 ml/well of RPMI 1640 with 10% heat inactivated fetal bovine serum (FBS). The cells were allowed to grow for approximately 40-48 hours before transfections were performed. At this point the cells were at confluency. Transfection solutions (0.1 ml/well for 96 well plates) were prepared by dilution of appropriate liposome samples to approximately 2 mM total lipid (for equal lipid transfection) into medium with 0.5% FBS. The plates were aspirated to remove medium and washed once with Dulbecco's phosphate buffered saline (PBS) followed by aspiration. After an addition of 1 mM CaCl₂ and 0.4 mM MgCl₂, the transfection solution was then added to the wells and incubated at 37° C. for 3 hours. After incubation, the wells were aspirated and a medium containing 10% heat inactivated FBS was added to each well. Because of the previously demonstrated silencing of transgenes, 5 mM of a histone deacetylase inhibitor, butyrate, was added to each well to enhance expression. After incubation at 37° C. in a cell culture incubator for 18-22 hours, the medium was aspirated and a 0.1 ml wash of Dulbecco's PBS was added. For quantifying EGFP gene expression, samples were then dissolved in a detergent and readings were taken for corrected total EGFP fluorescence in terms of the total number of live cells as previously described (Shangguan et al., Gene Therapy, 769-783, 2000).

### EXAMPLE 11

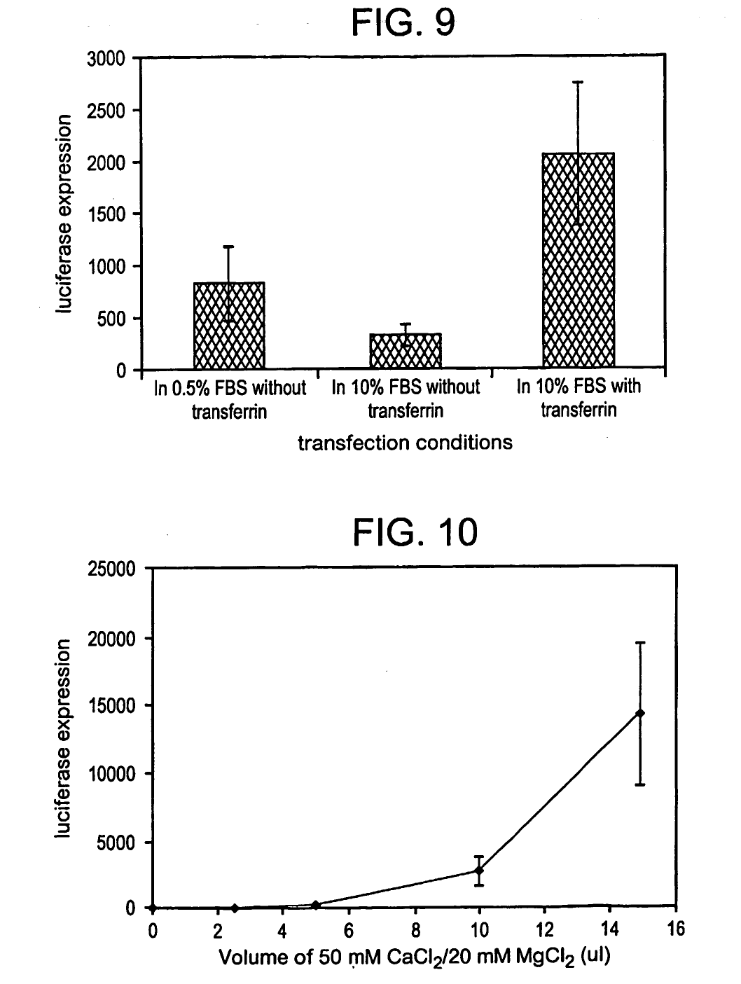

### Transfection Activity of N—C12-DOPE/DOPC (70:30) Liposomes in the Presence of 10% Serum, with and without Targeting via Transferrin (FIG. 9)

The N—C12-DOPE/DOPC (70:30) liposomes containing PGL-3 plasmid were made by the gel hydration method as set forth in Example 1. Transfections without transferrin were performed as described in example 10, except that in one of the transfection assays, 10% FBS instead of 0.5% FBS was used. For transferrin targeted transfection, the liposome samples were first mixed with equal volumes of a 2 mg/ml poly-lysin transferrin conjugate at a concentration of 20 mM for 10 minutes, and then this mixture was diluted 10 times with Hank's balanced salt solution (HBSS) without Ca²⁺/Mg²⁺ containing 10% FBS before being applied to the cells. The level of luciferase expression was determined by the Bright-glow luciferase assay (Clontech).

In the presence of 0.5% FBS, without transferrin, the sample showed significant transfection activity. In the presence of 10% FBS, the sample showed decreased but still considerable transfection. In the presence of 10% FBS, with transferrin, the sample showed a dramatic increase of transfection activity (FIG. 9).

### EXAMPLE 12

### Transfection Activity of N—C12-DOPE/DOPC (70:30) Liposomes at Physiological Ca²⁺/Mg²⁺ Concentrations (FIG. 10)

The N—C12-DOPE/DOPC (70:30) liposomes containing PGL-3 plasmid were made by the gel hydration method as set forth in Example 1. The transfections were performed as described in example 10, in the presence of 0.5% FBS and without targeting, except that various volumes of CaCl₂ and MgCl₂ solution were added to 500 μl of the transfection solution before their addition to the cells at 100 μl per well to test the Ca²⁺/Mg²⁺ dependence of the transfection activity. The level of luciferase expression was determined by the Bright-glow luciferase assay (Clontech). The N—C12-DOPE/DOPC (70:30) liposomes had transfection activity at physiological concentrations of Ca²⁺—Mg²⁺, i.e., about 1.2 mM Ca²⁺ and 0.8 mM Mg²⁺ (FIG. 10).

### EXAMPLE 13

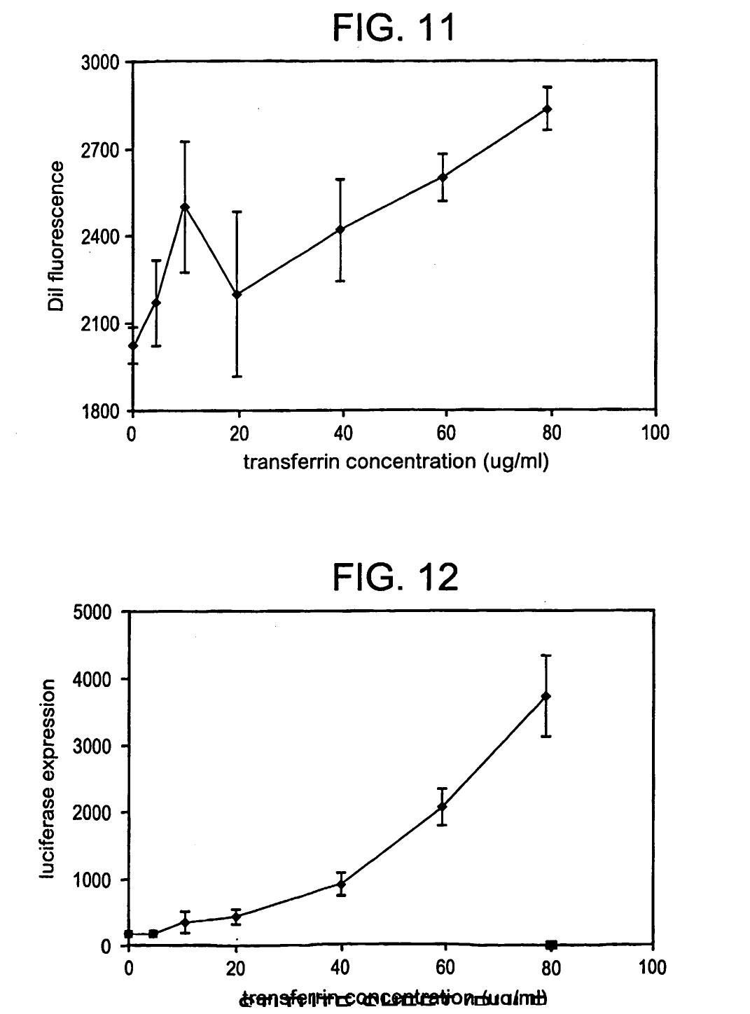

### Transferrin Mediated Binding of N—C12-DOPE/DOPC (70:30) Liposomes in 10% FBS (FIG. 11)

The N—C12-DOPE/DOPC (70:30) liposomes containing fluorescent lipid probe DiI at a 0.1% (wt %) concentration were prepared by the ethanol gel hydration method as set forth in Example 1. The liposomes were incubated with OVCAR-3 cells in the presence of 10% FBS and various concentrations of transferrin as described in Example 11. After a 3 hour incubation at 37° C., the cells were washed three times with PBS and dissolved in 1% C12E8. Cell associated DiI fluorescence was measured at an emission wavelength of 620 nm, with an excitation wavelength of 560 nm. Binding of the liposome sample showed a small increase with increasing transferrin concentration (FIG. 11).

### EXAMPLE 14

### Transferrin Mediated Transfection of N—C12-DOPE/DOPC (70:30) Liposomes in 10% FBS (FIG. 12)

The N—C12-DOPE/DOPC (70:30) liposomes containing PGL-3 plasmid were made by the gel hydration method as set forth in Example 1. The transfections were performed as described in Example 11, in the presence of 10% FBS and with various concentrations of transferrin for targeting. The level of luciferase expression was determined by the Bright-glow luciferase assay (Clontech). The liposome sample showed a transferrin dependent increase of transfection activity (FIG. 12).

### EXAMPLE 15

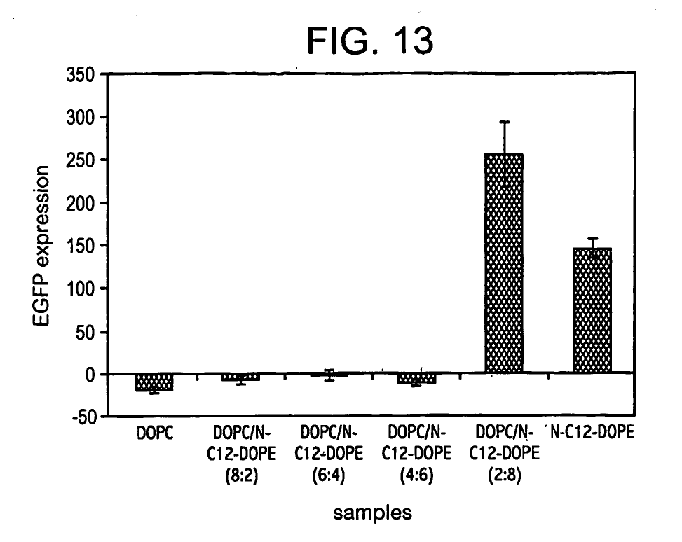

### Transfection Activity of Liposomes Containing DOPC, N—C12-DOPE, or DOPC/N—C12-DOPE at Various Ratios (FIG. 13)

The liposomes containing a EGFP plasmid DNA and the following lipids or lipid mixtures, including 100% DOPC, DOPC/N—C12-DOPE (8:2 molar ratio), DOPC/N—C12-DOPE (6:4 molar ratio), DOPC/N—C12-DOPE (4:6 molar ratio), DOPC/N—C12-DOPE (2:8 molar ratio), and 100% N—C12-DOPE, were made by the ethanol gel hydration method as set forth in Example 1. The transfection assay was performed as described in Example 10.

### EXAMPLE 16

### Encapsulation of Dextran

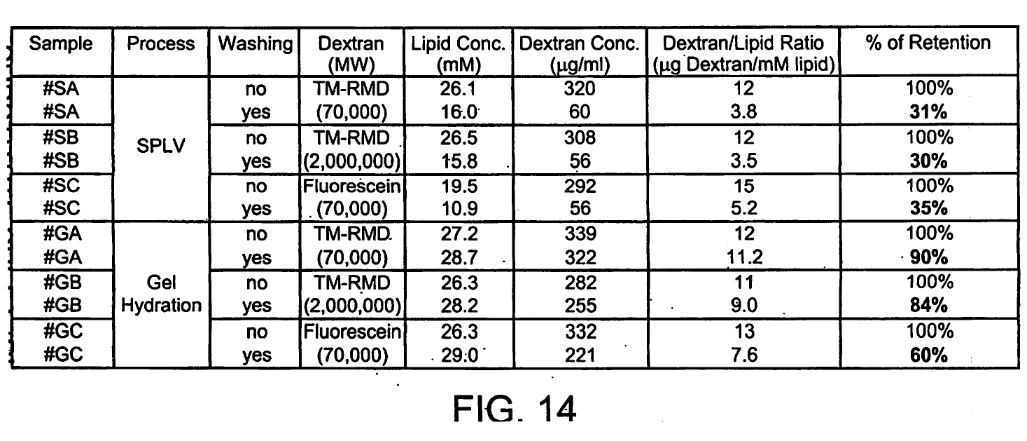

N—C12-DOPE/DOPC liposomes (70:30, molar ratio) were prepared by the gel hydration process (as set forth in Example 1) using 36.7 mg of N—C12-DOPE, 14.2 mg of DOPC and 100 μl of one of the following dextran stock solutions (5 mg/ml): tetramethyl rhodamine (MW 70,000), tetramethyl rhodamine (MW 2,000,000) or fluorescein (MW 70,000, lysine fixable). Conventional N—C12-DOPE/DOPC liposomes (70:30, molar ratio) were also prepared by the SPLV method: 1.13 ml of N—C12-DOPE/DOPC lipid mixtures (60 mM total lipid, 70:30 molar ratio) in chloroform were mixed with 100 μl of one of the following dextran stock solutions (5 mg/ml): tetramethyl rhodamine (MW 70,000), tetramethyl rhodamine (MW 2,000,000) or fluorescein (MW 70,000, lysine fixable). The mixture was sonicated briefly to form an emulsion. After most of the chloroform was removed by rotary evaporation at room temperature, 1:9 ml of a hydration buffer was added to the mixtures followed by additional 15 min of rotary evaporation. The unencapsulated material was removed by washing the liposomes three times via 10,000 g centrifugation. The dextran and lipid contents of each sample (FIG. 14) were determined using fluorescent measurement (excitation: 555 nm, emission: 580 nm) and a phosphate assay.

### EXAMPLE 17

### Evaluation of Captured Volumes and Lamellarity of Liposomes

To evaluate the captured volumes of the liposomes, the fluorescence intensity of an aqueous volume marker, sulphorodamine 101 (SR101), was measured. To remove non-entrapped material and ethanol, the liposomes were dialyzed against at least 1000-fold excess of a marker free buffer for at least 12 hrs with at least one buffer change. Then the liposomes were placed in microplates suitable for fluorescence measurements and diluted with a buffer containing detergent Triton X-100. Typical final lipid concentration in microplate wells was 0.5-2 mg/ml and the final detergent concentration was 1%. Under these conditions all liposomes were completely solubilized. The fluorescence intensity was measured using 560/20 and 620/40 nm bandpass excitation and emission filters, respectively. To evaluate the average number of bilayers in the liposomes, a lamellarity assay based on NBD-PE reduction by dithionite was used as described by McIntyre and Sleight (1991). All measurements of liposomes' captured volumes and lamellarity in Examples 18 through 25, 30 and 31 were conducted according to the procedures described in this example.

### EXAMPLE 18

### POPC

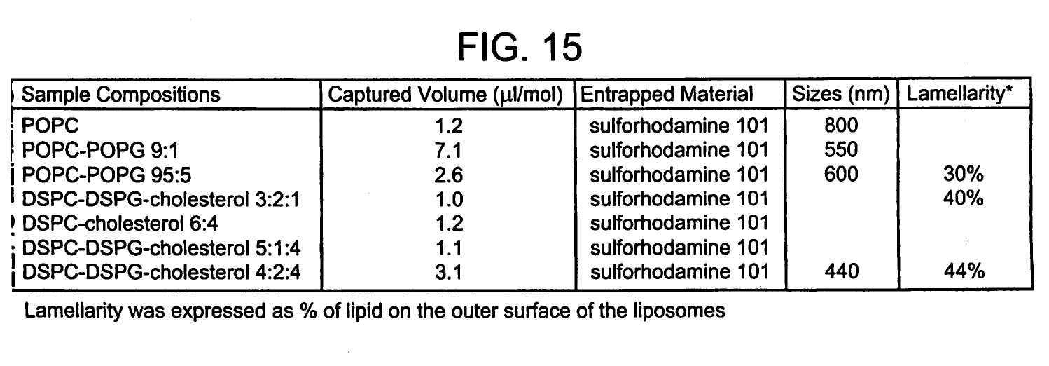

An amount of 346 mg of POPC was mixed with 346 mg of anhydrous EtOH, resulting in a clear solution. A Tris buffer, 100 mM, pH 7, containing 5 μM of sulforhodamine 101 as internal volume marker, was added to the clear solution in 10 μl aliquots upon rigorous vortexing. The clear solution became a viscous gel when a total of 40 μl of the buffer was added. Upon subsequent additions of the buffer, the gel became turbid. The sample became a liposome-like suspension when totally 80 μl of the buffer were added. The captured volume of the resultant liposomes, measured using SR101 fluorescence intensity, was 1.2 μl/μmol. Their average size, measured by dynamic light scattering, was 800 nm (FIG. 15).

### EXAMPLE 19

### POPC-POPG 9:1

An amount of 46 mg of POPC was mixed with 4 mg of POPG and dissolved in 50 mg of anhydrous EtOH. A 100 mM Tris buffer, pH 7, containing 5 μM sulforhodalamine 101 (SR101) as aqueous volume marker, was added to the lipid solution in 10 μl aliquots upon rigorous vortexing. The fluid solution became a viscous gel after addition of 20 μl of the buffer. The gel became a turbid liposome-like suspension after addition of a total of 70 μl of the buffer. The captured volume of the resultant liposomes was 7.1 μl/mol, as estimated using SR101 fluorescence. Their average diameter was 550 nm, as measured by dynamic light scattering (FIG. 15).

### EXAMPLE 20

### POPC-POPG 95:5

An amount of 47.5 mg of POPC was mixed with 2.5 mg of POPG and dissolved in 75 mg of anhydrous EtOH. An amount of 0.12 mg of NBD-PE was added to serve as a lamellarity probe. A 100 mM Tris buffer, containing 5 μM sulforhodamine 101 (SR101) as aqueous volume marker, was added to the lipid solution in 20 μl aliquots upon rigorous vortexing. The fluid solution became a viscous gel after addition of a total of 40 μl of the buffer. The gel became a turbid liposome-like suspension after addition of totally 120 μl of the buffer. The captured volume of the resultant liposomes was 2.6 μl/μmol, as estimated using SR101 fluorescence. About 30% of lipid were found on the outer shell of the liposomes, using a NBD-PE dithionite-reduction-based lamellarity assay. Average diameter of the liposomes was 600 nm as measured by dynamic light scattering (FIG. 15).

### EXAMPLE 21

### DSPC-DSPG-Cholesteral 3:2:1

Amounts of 27.6 mg of DSPC, 9 mg of DSPG and 9 mg of cholesterol were mixed with 50.3 mg of anhydrous EtOH at 55° C. A 50 mM MES+50 mM HEPES buffer, pH 7.2, containing 75 mM NaCl and 5 μM sulforhodamine 101, was added to the lipid solution in 10 μl aliquots upon rigorous vortexing. The sample and the titration buffer were maintained at 55° C. throughout the mixing process. The fluid solution became a viscous gel after an addition of 30 μl of the buffer. The gel became a turbid liposome-like suspension after an addition of totally 60 μl of the buffer. The captured volume of the resultant liposomes was 1 μl/μmol, as estimated using SR101 fluorescence. About 40% of the lipids was found on the outer shell of the liposomes, using a NBD-PE dithionite reduction based lamellarity assay. Average diameter of 75% of the liposomes was 330 nm, as measured by dynamic light scattering, with the remaining 15% larger than 5 μm, as they did not pass through 5 μm pore size filter (FIG. 15).

### EXAMPLE 22

### DMPC-Cholesterol-PA 4:1:0.2

Amounts of 90 mg of DMPC, 13 mg of cholesterol and 2.5 mg of PA were dissolved in 115.5 mg of anhydrous EtOH. When the solution was rapidly mixed with 230 μl of a 10 mM borate buffer, pH 9.7, containing 140 mM NaCl and β-amyloid peptide at 5 mg/ml, a viscous gel was obtained. The gel was hydrated by 2 ml of the same buffer, but containing no β-amyloid peptide, and the sample was dialyzed against 1 L of the same buffer to remove ethanol and non-entrapped peptide. The resultant liposomes captured 40% of the peptide added in the first step.

### EXAMPLE 23

### DSPC-Cholesterol 6:4

Amounts of 78 mg of DSPC and 25 mg of cholesterol were dissolved in 103 mg of anhydrous EtOH. A 100 mM Tris buffer, pH 7, containing 5 uM sulforhodamine 101, was added to the lipid solution in 10 μl aliquots upon rigorous vortexing. The sample and the titration buffer were maintained at 55° C. throughout the mixing process. The fluid solution became a viscous gel after an addition of 40 μl of the buffer. The gel became a turbid liposome-like suspension after an addition of totally 70 μl of the buffer. After an addition of 500 more μl of the buffer, the liposomes were cooled to room temperature upon vortexing, and dialyzed against 1 L of a 100 mM Tris buffer, pH 7, buffer overnight to remove ethanol and nonentrapped material. The captured volume of the resultant liposomes was 1.2 uL/umol, as estimated using SR 101 fluorescence (FIG. 15)

### EXAMPLE 24

### DSPC-DSPG-Cholesterol 5:1:4

Amounts of 20.3 mg of DSPC, 3.3 mg of DSPG and 6.3 mg of cholesterol were dissolved in 30 mg of anhydrous EtOH at 60° C. A 100 mM Tris buffer, pH 7, containing 5 μM sulforhodamine 101, was added to the lipid solution in 5 μl aliquots upon rigorous vortexing. The sample and the titration buffer were maintained at 60° C. throughout the mixing process. The fluid solution became a viscous gel after an addition of 5 μl of the buffer. The gel became a turbid liposome-like suspension after adding a total of 20 μl of the buffer. After mixing with an additional 500 μl of the buffer, the liposomes were cooled to room temperature upon vortexing, and dialyzed against 1 L of a 100 mM Tris buffer, pH 7, for 12 hours to remove ethanol and nonentrapped material. The captured volume of the resultant liposomes was 1.1 uL/umol, as estimated using SR 101 fluorescence. It was found that 78% of the liposomes were less than 1 μm in diameter, as determined by using a filter with 1 μm pore size (FIG. 15).

### EXAMPLE 25

### DSPC-DSPG-Cholesterol 4:2:4

Amounts of 42.8 mg of DSPC, 21.7 mg of DSPG and 21 mg of cholesterol were dissolved in 132 mg of anhydrous EtOH at 60° C. An amount of 0.17 mg of NBD-PE was added to serve as a lamellarity probe. A 100 mM Tris buffer, pH 7, containing 5 μM sulforhodamine 101, was added to the lipid solution in 10 μl aliquots upon rigorous vortexing. The sample and the titration buffer were maintained at 60° C. throughout the mixing process. The fluid solution became a viscous gel after an addition of 40 μl of the buffer. The gel became a turbid liposome-like suspension after an addition of totally 130 μl of the buffer. After adding an additional 370 μl of the buffer, the liposomes were cooled to room temperature upon vortexing, and dialyzed against 1 L of 100 mM Tris buffer, pH 7, overnight to remove ethanol and nonentrapped material. The captured volume of the resultant liposomes was 3.1 μl/μmol, as estimated using SR 101 fluorescence, 44% of lipid was found on the outer shell of the liposomes, using a NBD-PE dithionite reduction based lamellarity assay. Average diameter of the liposomes was 440 nm, as measured by dynamic light scattering (FIG. 15).

### EXAMPLE 26

### Ternary Phase Diagram of a POPC/Ethanol/Aqueous Medium System

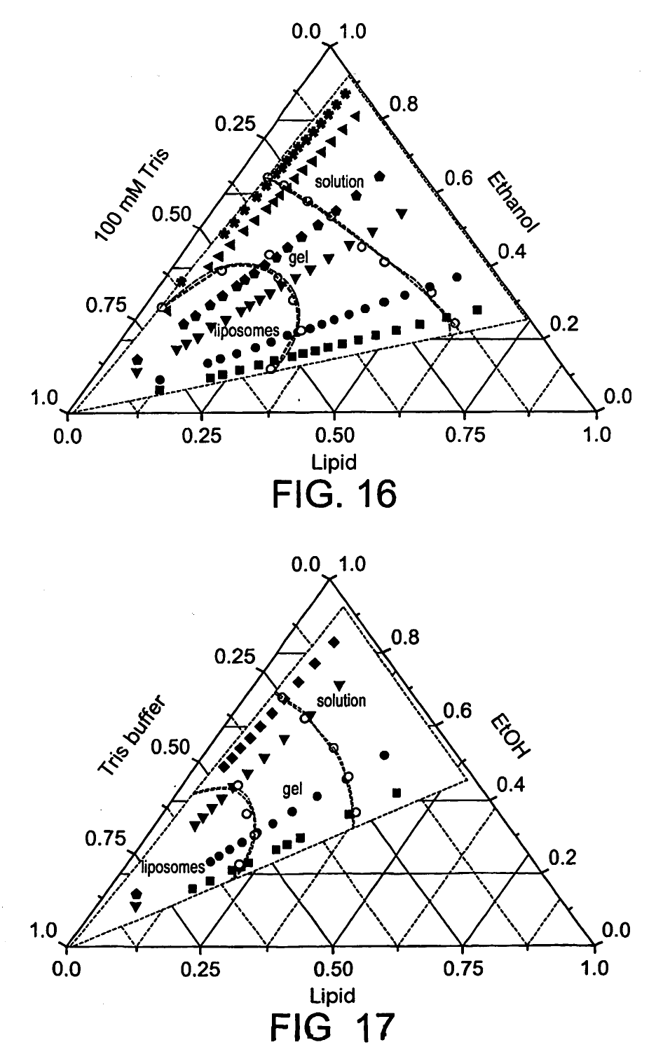

The ternary phase diagram of a lipid/water-miscible organic solvent/aqueous medium system was produced, wherein the lipid was POPC, the water-miscible organic solvent was ethanol and the aqueous medium was a 100 mM Tris buffer. Varying amounts (30-80 mg) of POPC were dissolved in anhydrous ethanol to form a lipid solution at concentrations of 10%, 20%, 30%, 50%, 60% and 70% (wt/wt). The 100 mM Tris buffer was added to the lipid solution in 10 μl increments upon vigorous vortexing. Appearance of the samples after each titration step was recorded. With the incremental addition of the 100 mM Tris buffer, the lipid solution first turned into a gel, which turned into a liposome suspension upon further incremental addition of the 100 mM Tris buffer. A ternary phase diagram was constructed mapping the locations of liquid, gel and liposomal states of the samples based on their visual appearance. The boundary between the solution zone and the gel zone was as indicated by the open circles and dotted line in the ternary phase diagram of FIG. 16. Additional amounts of the 100 mM Tris buffer were added to the gel with mixing to form liposomes. The boundary between the gel zone and the liposome zone was as indicated by the open circles and lines in the ternary phase diagram of FIG. 16. In six different preparations (represented by six different symbols: stars, triangles, pentagons, inverted triangles, circles and squares), the 100 mM Tris buffer was added in 10 μl increments as represented by the individual symbols in FIG. 16.

### EXAMPLE 27

### Ternary Phase Diagram of a POPC-POPG (95:5)/Ethanol/Aqueous Medium System

The ternary phase diagram of a lipid/water-miscible organic solvent/aqueous medium system was produced, wherein the lipids were POPC and POPG in a 95:5 molar ratio, the water-miscible organic solvent was ethanol and the aqueous medium was a 100 mM Tris buffer. Varying amounts (30-80 mg) of the POPC:POPG mixture (95:5) were dissolved in anhydrous ethanol to form a lipid solution at concentrations of 10%, 20%, 30% and 50% (wt/wt). The 100 mM Tris buffer was added to the lipid solution in 10 μl increments upon vigorous vortexing. Appearance of the samples after each titration step was recorded. With the incremental addition of the 100 mM Tris buffer, the lipid solution first turned into a gel, which turned into a liposome suspension upon further incremental addition of the 100 mM Tris buffer. A ternary phase diagram was constructed mapping the locations of liquid, gel and liposomal states of the samples based on their visual appearance. The boundary between the solution zone and the gel zone was as indicated by the open circles and the dotted line in the ternary phase diagram of FIG. 17. Additional amounts of the 100 mM Tris buffer were added to the gel with mixing to form liposomes. The boundary between the gel zone and the liposome zone was as indicated by the open circles and the dotted line in the ternary phase diagram of FIG. 17. In four different preparations (represented by four different symbols: grey squares, dark triangles, grey circles and dark squares), the 100 mM Tris buffer was added in 10 μl increments as represented by the individual symbols in FIG. 17.

### EXAMPLE 28

### Ternary Phase Diagram of a POPC-POPG (9:1)/Ethanol/Aqueous Medium System

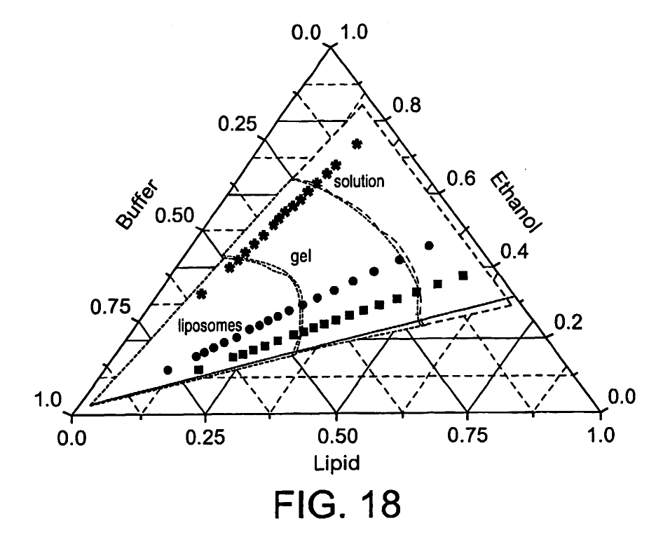

The ternary phase diagram of a lipid/water-miscible organic solvent/aqueous medium system was produced, wherein the lipids were POPC and POPG in a 9:1 molar ratio, the water-miscible organic solvent was ethanol and the aqueous medium was a 100 mM Tris buffer. Varying amounts (30-80 mg) of the POPC:POPG mixture (9:1) were dissolved in anhydrous ethanol to form a lipid solution at concentrations of 20%, 30%, 50% and 60% (wt/wt). The 100 mM Tris buffer was added to the lipid solution in 10 μl increments upon vigorous vortexing. Appearance of the samples after each titration step was recorded. With the incremental addition of the 100 mM Tris buffer, the lipid solution first turned into a gel, which turned into a liposome suspension upon further incremental addition of the 100 mM Tris buffer. A ternary phase diagram was constructed mapping the locations of liquid, gel and liposomal states of the samples based on their visual appearance. The boundary between the solution zone and the gel zone was as indicated by the dashed line in the ternary phase diagram of FIG. 18. Additional amounts of the 100 mM Tris buffer were added to the gel with mixing to form liposomes. The boundary between the gel zone and the liposome zone was as indicated by the dotted line in the ternary phase diagram of FIG. 18. In three different preparations (represented by three different symbols: stars, circles and squares), the 100 mM Tris buffer was added in 10 μl increments as represented by the individual symbols in FIG. 18.

### EXAMPLE 29

### Dependence of Liposome Entrapment Efficiency on the Gel Composition

Two similar lipid-ethanol mixtures, containing 39 mg of POPC, 5 mg POPG and 44 mg of anhydrous ethanol were prepared in separate sample tubes (POPC/POPG 9:1 (mol/mol), lipid/ethanol 1:1 (wt/wt)). One mixture was rapidly mixed 23 mg of 100 mM Tris buffer (pH 7), containing 5 mM of SR101 (as an aqueous volume marker) (sample 1), another one was mixed with 69 mg of the same buffer (sample 2). In both samples formation of gel was observed. The gel in both samples was hydrated with 300 mg of the same buffer, containing no SR 101. Upon hydration the gel transformed into the liposomal suspension. The samples were dialyzed against 2000× excess of 100 mM Tris buffer to remove ethanol and non-encapsulated SR11. Encapsulation efficiency of resultant liposomes was evaluated by measuring fluorescence intensity of aqueous volume marker SR11. The entrapment efficiency of the liposomes in sample 1, prepared from the gel with low content of aqueous phase was only 10%, while the entrapment efficiency of the liposomes in sample 2, prepared from the gel containing more aqueous phase, was 67%.

### EXAMPLE 30

### Preparation of Liposomes by Converting Gel to Waxy Substance

Amounts of 27.8 mg of DSPC, 8.9 mg of DSPG, 8.86 mg of cholesterol and 0.11 mg of NBD-PE (as a lamellarity probe) were dissolved in ethanol at 60° C. 25 μl of 50 mM MES+50 mM HEPES buffer (pH 7.2), containing 75 mM NaCl and 5 μM sulforhodamine 101, i.e., SR 101 (as aqueous volume marker), at 60° C. were rapidly mixed with solution of lipids in ethanol, maintained at the same temperature. Formation of gel was observed. The gel was cooled to room temperature upon rigorous vortexing. Upon cooling the gel solidified and became a solid waxy substance. 1 ml of the same buffer, containing no sulforhodamine 101, was added to the waxy substance at room temperature and rigorously vortexed. A homogeneous suspension of liposomes was obtained. 73% of the resultant liposomes passed through a filter with 5 μm pore size, and their average diameter was 120 nm, as measured by dynamic light scattering. The entrapment efficiency of the liposomes was over 80%, as evaluated from measurements of sulforhodamine 101 fluorescence.

### EXAMPLE 31

### Example of Transitional Curdy Substance Stage

Amounts of 43 mg of DSPC, 21.8 mg of DSPG, 21 mg of cholesterol and 0.15 mg of NBD-PE (as a lamellarity probe) were dissolved in 132 mg of ethanol at 60° C. 100 mM Tris buffer (pH 7), containing 5 uM sulforhodamine 101 (as aqueous volume marker) was added in 10 μl aliquots into lipid solution in ethanol upon rigorous vortexing. Both buffer and sample were maintained at 60° C. throughout the titration process. Upon addition of 30 μl of the buffer the sample became a viscous gel. Upon addition of a total of 90 μl of the buffer, the gel transformed into a curdy substance. Upon addition of a total of 130 μl of the buffer, the sample became a liposomal suspension. After addition of total 500 μl of the buffer, the sample was dialyzed and analyzed for captured volumes of and lamellarity of the liposomes as described in Example 18. The captured volumes of the resultant liposomes were 3.1 μl/μmol, and about 44% of lipid were found on the outer shell of the liposomes. Average diameters of the liposomes were 330 nm (74%) and 615 nm (26%) as measured by dynamic light scattering.

What is claimed is:

1. A method for preparing liposomes, said method comprising the following steps:
(I) mixing at least one liposome-forming lipid, a water-miscible organic solvent and aqueous medium Y to form a gel or liquid containing gel particles without creation of any gas/aqueous phase boundary; wherein if the gel or liquid containing gel particles contains at least one acidic phospholipid, the content of the at least one acidic phospholipid is about 30% to about 100% by weight of the lipid(s) in the gel or liquid containing gel particles; and thereafter
(II) (a) mixing the gel or liquid containing gel particles with aqueous medium Z1 to directly form the liposomes;
(b) (i) mixing the gel or liquid containing gel particles with aqueous medium Z1 to form a curd or curdy substance; and
(ii) mixing the curd or curdy substance with aqueous medium Z2 to directly form the liposomes; or
(c) (i) cooling the gel or liquid containing gel particles to form a waxy substance; and
(ii) mixing the waxy substance with aqueous medium Z1 to directly form the liposomes;
wherein aqueous media Y, Z1 and Z2 are the same or different.

2. A method for preparing liposomes containing at least one biologically active substance encapsulated therein, said method comprising the following steps:
(I) (A) mixing at least one liposome-forming lipid, the at least one biologically active substance, a water-miscible organic solvent and aqueous medium Y to form a gel or liquid containing gel particles without creation of any gas/aqueous phase boundary; wherein if the gel or liquid containing gel particles contains at least one acidic phospholipid, the content of the at least one acidic phospholipid is about 30% to about 100% by weight of the lipid(s) in the gel or liquid containing gel particles; or
(B) mixing at least one liposome-forming lipid, a water-miscible organic solvent and aqueous medium Y to form a gel or liquid containing gel particles without creation of any gas/aqueous phase boundary; wherein if the gel or liquid containing gel particles contains at least one acidic phospholipid, the content of the at least one acidic phospholipid is about 30% to about 100% by weight of the lipid(s) in the gel or liquid containing gel particles;
(II) (A) mixing the gel or liquid containing gel particles of step (I)(A) with aqueous medium Z1 to directly form the liposomes containing the at least one biologically active substance encapsulated in the liposomes;
(B) (i) mixing the gel or liquid containing gel particles of step (I)(A) with aqueous medium Z1 to form a curd or curdy substance; and
(ii) mixing the curd or curdy substance with aqueous medium Z2 to directly form the liposomes containing the at least one biologically active substance encapsulated in the liposomes;
(C) (i) cooling the gel or liquid containing gel particles of step (I)(A) to form a waxy substance; and
(ii) mixing the waxy substance with aqueous medium Z1 to directly form the liposomes;
(D) mixing the gel or liquid containing gel particles of step (I) (B) with aqueous medium Z1 and the at least one biologically active substance to directly form the liposomes containing the at least one biologically active substance encapsulated in the liposomes;
(E) (i) mixing the gel or liquid containing gel particles of step (I)(B) with aqueous medium Z1 and the at least one biologically active substance to form a curd or curdy substance; and
(ii) mixing the curd or curdy substance with aqueous medium Z2 to directly form the liposomes containing the at least one biologically active substance encapsulated in the liposomes;
(F) (i) mixing the gel or liquid containing gel particles of step (I)(B) with aqueous medium Z1 to form a curd or curdy substance; and
(ii) mixing the curd or curdy substance with aqueous medium Z2 and the at least one biologically active substance to directly form the liposomes containing the at least one biologically active substance encapsulated in the liposomes; or
(G) (i) cooling the gel or liquid containing gel particles of step (I)(B) to form a waxy substance; and
(ii) mixing the waxy substance with aqueous medium Z1 and the at least one biologically active substance to directly form the liposomes;
wherein aqueous media Y, Z1 and Z2 are the same or different.

3. The method of claim 2, wherein
in step (II)(A) or (II)(B) the gel or liquid containing the gel particles are mixed with aqueous medium Z1 and the at least one biologically active substance; or
in step (II)(C)(ii) the curd or curdy substance is mixed with aqueous medium Z2 and the at least one biologically active substance.

4. The method of claim 2, wherein the organic solvent is selected from the group consisting of acetaldehyde, acetone, acetonitrile, allyl alcohol, allylamine, 2-amino-1-butanol, 1-aminoethanol, 2-aminoethanol, 2-amino-2-ethyl-1,3-propanediol, 2-amino-2-methyl-1-propanol, 3-aminopentane, N-(3-aminopropyl)morpholine, benzylamine, bis(2-ethoxyethyl)ether, bis(2-hydroxyethyl)ether, bis(2-hydropropyl)ether, bis(2-methoxyethyl)ether, 2-bromoethanol, meso-2,3-butanediol, 2-(2-butoxyethoxy)-ethanol, butylamine, sec-butylamine, tert-butylamine, 4-butyrolacetone, 2-chloroethanol, 1-chloro-2-propanol, 2-cyanoethanol, 3-cyanopyridine, cyclohexylamine, diethylamine, diethylenetriamine, N,N-diethylformamide, 1,2-dihydroxy-4-methylbenzene, N,N-dimethylacetamide, N,N-dimethylformaide, 2,6-dimethylmorpholine, 1,4-dioxane, 1,3-dioxolane, dipentaerythritol, ethanol, 2,3-epoxy-1-propanol, 2-ethoxyethanol, 2-(2-ethoxyethoxy)-ethanol, 2-(2-ethoxyethoxy)-ethyl acetate, ethylamine, 2-(ethylamino)ethanol, ethylene glycol, ethylene oxide, ethylenimine, ethyl(−)-lactate, N-ethylmorpholine, ethyl-2-pyridine-carboxylate, formamide, furfuryl alcohol, furfurylamine, glutaric dialdehyde, glycerol, hexamethylphosphor-amide, 2,5-hexanedione, hydroxyacetone, 2-hydroxyethyl-hydrazine, N-(2-hydroxyethyl)-morpholine, 4-hydroxy-4-methyl-2-pentanone, 5-hydroxy-2-pentanone, 2-hydroxypropionitrile, 3-hydroxypropionitrile, 1-(2-hydroxy-1-propoxy)-2-propanol, isobutylamine, isopropylamine, 2-isopropylamino-ethanol, 2-mercaptoethanol, methanol, 3-methoxy-1-butanol, 2-methoxyethanol, 2-(2-methoxyethoxy)-ethanol, 1-methoxy-2-propanol, 2-(methylamino)-ethanol, 1-methylbutylamine, methylhydrazine, methyl hydroperoxide, 2-methylpyridine, 3-methylpyridine, 4-methylpyridine, N-methylpyrrolidine, N-methyl-2-pyrrolidinone, morpholine, nicotine, piperidine, 1,2-propanediol, 1,3-propanediol, 1-propanol, 2-propanol, propylamine, propyleneimine, 2-propyn-1-ol, pyridine, pyrimidine, pyrrolidine, 2-pyrrolidinone and quinoxaline.

5. The method of claim 4, wherein the organic solvent is acetonitrile, acetone, methanol, ethanol, 1-propanol, 2-propanol, ethylene glycol or propylene glycol.

6. The method of claim 5, wherein the organic solvent is ethanol, 1-propanol or 2-propanol.

7. The method of claim 6, wherein the organic solvent is ethanol.

8. The method of claim 5, wherein the organic solvent is acetone.

9. The method of claim 2, wherein aqueous medium Y, aqueous medium Z1 and/or aqueous medium Z2 is an aqueous buffer.

10. The method of claim 2, wherein the gel or liquid containing the gel particles and aqueous medium Z1 are mixed in step (II) by adding aqueous medium Z1 to the gel or liquid containing the gel particles.

11. The method of claim 2, wherein the gel or liquid containing the gel particles and aqueous medium Z1 are mixed in step (II) by adding the gel or liquid containing the gel particles into aqueous medium Z1.

12. The method of claim 2, wherein the at least one biologically active substance is at least one nucleic acid, protein, peptide, pharmaceutical agent or diagnostic agent.

13. The method of claim 12, wherein the at least one biologically active substance is at least one nucleic acid.

14. The method of claim 13, wherein the at least one nucleic acid is a DNA.

15. The method of claim 13, wherein the at least one nucleic acid is a plasmid DNA of up to about 20 kb in size.

16. The method of claim 13, wherein the at least one nucleic acid is a RNA.

17. The method of claim 15, wherein the at least one nucleic acid is an oligonucleotide of about 5 to about 500 bases in size.

18. The method of claim 2, wherein the at least one biologically active substance is at least one protein or antigen structurally sensitive to dehydration.

19. The method of claim 2, wherein the at least one biologically active substance is at least one pharmaceutical agent selected from the group consisting of anti-tumor agents, anti-neoplastic agents, anti-microbial agents, anti-viral agents, antihypertensive agents, anti-inflammatory agents, bronchodilators, local anesthetics and immunosuppressants.

20. The method of claim 19, wherein the at least one pharmaceutical agent is selected from the group consisting of anti-bacterial agents and anti-fungal agents.

21. The method of claim 20, wherein the at least one pharmaceutical agent is selected from the group consisting of anti-fungal agents.

22. The method of claim 21, wherein the at least one pharmaceutical agent is a bioactive lipid.

23. The method of claim 19, wherein the at least one pharmaceutical agent is selected from the group consisting of anti-neoplastic agents.

24. The method of claim 2, wherein the at least one biologically active substance is an antibody or toxoid.

25. The method of claim 2, wherein the at least one liposome-forming lipid is selected from the group consisting of phospholipids, glycolipids and sphingolipids.

26. The method of claim 23, wherein the at least one liposome-forming lipid is selected from the group consisting of phospholipids.

27. The method of claim 24, wherein the at least one liposome-forming lipid is selected from the group consisting of phosphatidylcholine, phosphatidylserine, phosphatidylinositol, phosphatidylglycerol, diphosphatidylglycerol and N-acyl phosphatidylethanolamine.

28. The method of claim 27, wherein the at least one liposome-forming lipid is selected from the group consisting of dioleoyl phosphatidylcholine, dipalmitoyl phosphatidylcholine, distearoyl phosphatidylcholine, dimyristoyl phosphatidylcholine, 1-palmitoyl-2-oleoyl-sn-glycero-3-phosphocholine, 1-oleoyl-2-palmitoyl-sn-glycero-3-phosphocholine, 1,2-dioleoyl-sn-glycero-3-[phospho-rac-(1-glycerol)], 1,2-dipalmitoyl-sn-glycero-3-[phospho-rac-(1-glycerol)], 1,2-distearoyl-sn-glycero-3-[phospho-rac-(1-glycerol)], 1,2-dimyristoyl-sn-glycero-3-[phospho-rac-(1-glycerol)], 1-palmitoyl-2-oleoyl-sn-glycero-3-[phospho-rac-(1-glycerol)], 1-oleoyl-2-palmitoyl-sn-glycero-3-[phospho-rac-(1-glycerol)], N-decanoyl phosphatidylethanolamine, N-undecanoyl phosphatidylethanolamine, N-dodecanoyl phosphatidylethanolamine, N-tridecanoyl phosphatidylethanolamine, and N-tetradecanoyl phosphatidylethanolamine.

29. The method of claim 2, wherein the gel or liquid containing gel particles further comprises a sterol.

30. The method of claim 29, wherein the sterol is cholesterol.

31. The method of claim 2, wherein the at least one liposome-forming lipid is a N-acyl phosphatidylethanolamine.

32. The method of claim 31, wherein the N-acyl phosphatidylethanolamine. is 1,2-dioleoyl-sn-glycero-N-dodecanoyl-3-phosphoethanolamine.

33. The method of claim 2, wherein in the gel or the liquid containing gel particles of step (I), the amount of the at least one liposome-forming lipid ranges from about 1% by weight of the gel or the liquid containing gel particles to the hydration limit of the at least one liposome-forming lipid in water, wherein the hydration limit is the maximum amount of the at least one liposome-forming lipid in a given amount of water that would keep the at least one liposome-forming lipid in a liposomal state; provided that when the at least one liposome-forming lipid is at least one acidic phospholipid, the amount of the at least one liposome-forming lipid ranges from about 30% to about 100% by weight of the lipid(s) in the gel or the liquid containing gel particles.

34. The method of claim 2, wherein in the gel or the liquid containing gel particles of step (I), an amount of the at least one liposome-forming lipid ranges from about 5% to about 80% by weight of the gel or the liquid containing gel particles; provided that when the at least one liposome-forming lipid is at least one acidic phospholipid, the amount of the at least one liposome-forming lipid ranges from about 30% to about 100% by weight of the lipid(s) in the gel or the liquid containing gel particles.

35. The method of claim 34, wherein in the gel or the liquid containing gel particles of step (I), said amount of the at least one liposome-forming lipid ranges from about 10% to about 80% by weight of the gel or the liquid containing gel particles.

36. The method of claim 35, wherein in the gel or the liquid containing gel particles of step (I), said amount of the at least one liposome-forming lipid ranges from about 15% to about 80% by weight of the gel or the liquid containing gel particles.

37. The method of claim 36, wherein in the gel or the liquid containing gel particles of step (I), said amount of the at least one liposome-forming lipid ranges from about 20% to about 80% by weight of the gel or the liquid containing gel particles.

38. The method of claim 37, wherein in the gel or the liquid containing gel particles of step (I), said amount of the at least one liposome-forming lipid ranges from about 30% to about 80% by weight of the gel or the liquid containing gel particles.

39. The method of claim 38, wherein in the gel or the liquid containing gel particles of step (I), said amount of the at least one liposome-forming lipid ranges from about 40% to about 80% by weight of the gel or the liquid containing gel particles.

40. The method of claim 39, wherein in the gel or the liquid containing gel particles of step (I), said amount of the at least one liposome-forming lipid ranges from about 50% to about 80% by weight of the gel or the liquid containing gel particles.

41. The method of claim 34, wherein in the gel or the liquid containing gel particles of step (I), said amount of the at least one liposome-forming lipid ranges from about 10% to about 70% by weight of the gel or the liquid containing gel particles.

42. The method of claim 41, wherein in the gel or the liquid containing gel particles of step (I), said amount of the at least one liposome-forming lipid ranges from about 20% to about 60% by weight of the gel or the liquid containing gel particles.

43. The method of claim 42, wherein in the gel or the liquid containing gel particles of step (I), said amount of the at least one liposome-forming lipid ranges from about 30% to about 50% by weight of the gel or the liquid containing gel particles.

44. The method of claim 43, wherein in the gel or the liquid containing gel particles of step (I), said amount of the at least one liposome-forming lipid is about 45% by weight of the gel or the liquid containing gel particles.

45. The method of claim 2, wherein steps (I) and (II) are conducted by
(I) (A) (i) mixing the at least one liposome-forming lipid, the at least one biologically active substance, the water-miscible organic solvent and aqueous medium Y to form a clear gel or a liquid containing clear gel particles without the creation of any gas/aqueous phase boundary; and
(ii) mixing the clear gel or liquid containing clear gel particles with additional aqueous medium Y to form a cloudy gel or a liquid containing cloudy gel particles; or
(B) (i) mixing the at least one liposome-forming lipid, the at least one biologically active substance, the water-miscible organic solvent and aqueous medium Y to form a clear gel or a liquid containing clear gel particles without the creation of any gas/aqueous phase boundary; and
(ii) mixing the clear gel or the liquid containing clear gel particles with additional aqueous medium Y to form a cloudy gel or a liquid containing cloudy gel particles; and thereafter
(II) (A) mixing the cloudy gel or the liquid containing cloudy gel particles of step (I)(A) with aqueous medium Z1 to directly form the liposomes containing the at least one biologically active substance encapsulated in the liposomes;
(B) (i) mixing the cloudy gel or the liquid containing cloudy gel particles of step (I)(A) with aqueous medium Z1 to form a curd or curdy substance; and
(ii) mixing the curd or curdy substance with aqueous medium Z2 to directly form the liposomes containing the at least one biologically active substance encapsulated in the liposomes;
(C) (i) cooling the cloudy gel or liquid containing cloudy gel particles of step (I)(A) to form a waxy substance; and
(ii) mixing the waxy substance with aqueous medium Z1 to directly form the liposomes;
(D) mixing the cloudy gel or liquid containing cloudy gel particles of step (I)(B) with aqueous medium Z1 and the at least one biologically active substance to directly form the liposomes containing the at least one biologically active substance encapsulated in the liposomes;
(E) (i) mixing the cloudy gel or liquid containing cloudy gel particles of step (I)(B) with aqueous medium Z1 and the at least one biologically active substance to form a curd or curdy substance; and
(ii) mixing the curd or curdy substance with aqueous medium Z2 to directly form the liposomes containing the at least one biologically active substance encapsulated in the liposomes;
(F) (i) mixing the cloudy gel or liquid containing cloudy gel particles of step (I)(B) with aqueous medium Z1 to form a curd or curdy substance; and
(ii) mixing the curd or curdy substance with aqueous medium Z2 and the at least one biologically active substance to directly form the liposomes containing the at least one biologically active substance encapsulated in the liposomes; or
(G) (i) cooling the cloudy gel or liquid containing cloudy gel particles of step (I)(B) to form a waxy substance; and
(ii) mixing the waxy substance with aqueous medium Z1 and the at least one biologically active substance to directly form the liposomes.

46. The method of claim 2, wherein after step (II) the liposomes are washed with an aqueous medium by centrifugation, gel filtration or dialysis.

47. The method of claim 1, wherein the organic solvent is selected from the group consisting of acetaldehyde, acetone, acetonitrile, allyl alcohol, allylamine, 2-amino-1-butanol, 1-aminoethanol, 2-aminoethanol, 2-amino-2-ethyl-1,3-propanediol, 2-amino-2-methyl-1-propanol, 3-aminopentane, N-(3-aminopropyl)morpholine, benzylamine, bis(2-ethoxyethyl)ether, bis(2-hydroxyethyl)ether, bis(2-hydropropyl)ether, bis(2-methoxyethyl)ether, 2-bromoethanol, meso-2,3-butanediol, 2-(2-butoxyethoxy)-ethanol, butylamine, sec-butylamine, tert-butylamine, 4-butyrolacetone, 2-chloroethanol, 1-chloro-2-propanol, 2-cyanoethanol, 3-cyanopyridine, cyclohexylamine, diethylamine, diethylenetriamine, N,N-diethylformamide, 1,2-dihydroxy-4-methylbenzene, N,N-dimethylacetamide, N,N-dimethylformaide, 2,6-dimethylmorpholine, 1,4-dioxane, 1,3-dioxolane, dipentaerythritol, ethanol, 2,3-epoxy-1-propanol, 2-ethoxyethanol, 2-(2-ethoxyethoxy)-ethanol, 2-2-ethoxyethoxy)-ethyl acetate, ethylamine, 2-(ethylamino)ethanol, ethylene glycol, ethylene oxide, ethylenimine, ethyl(−)-lactate, N-ethylmorpholine, ethyl-2-pyridine-carboxylate, formamide, furfuryl alcohol, furfurylamine, glutaric dialdehyde, glycerol, hexamethylphosphor-amide, 2,5-hexanedione, hydroxyacetone, 2-hydroxyethyl-hydrazine, N-(2-hydroxyethyl)-morpholine, 4-hydroxy-4-methyl-2-pentanone, 5-hydroxy-2-pentanone, 2-hydroxypropionitrile, 3-hydroxypropionitrile, 1-(2-hydroxy-1-propoxy)-2-propanol, isobutylamine, isopropylamine, 2-isopropylamino-ethanol, 2-mercaptoethanol, methanol, 3-methoxy-1-butanol, 2-methoxyethanol, 2-(2-methoxyethoxy)-ethanol, 1-methoxy-2-propanol, 2-(methylamino)-ethanol, 1-methylbutylamine, methylhydrazine, methyl hydroperoxide, 2-methylpyridine, 3-methylpyridine, 4-methylpyridine, N-methylpyrrolidine, N-methyl-2-pyrrolidinone, morpholine, nicotine, piperidine, 1,2-propanediol, 1,3-propanediol, 1-propanol, 2-propanol, propylamine, propyleneimine, 2-propyn-1-ol, pyridine, pyrimidine, pyrrolidine, 2-pyrrolidinone and quinoxaline.

48. The method of claim 47, wherein the organic solvent is acetonitrile, acetone or a C₁-C₃ alcohol.

49. The method of claim 48, wherein the organic solvent is methanol, ethanol, 1-propanol, 2-propanol, ethylene glycol or propylene glycol.

50. The method of claim 49, wherein the organic solvent is ethanol, 1-propanol or 2-propanol.

51. The method of claim 50, wherein the organic solvent is ethanol.

52. The method of claim 47, wherein the organic solvent is acetone.

53. The method of claim 1, wherein aqueous medium Y, aqueous medium Z1 and/or aqueous medium Z2 is an aqueous buffer.

54. The method of claim 1, wherein the gel or the liquid containing gel particles and aqueous medium Z1 are mixed in step (II) by adding aqueous medium Z1 to the gel or the liquid containing gel particles.

55. The method of claim 1, wherein the gel or the liquid containing gel particles and aqueous medium Z1 are mixed in step (II) by adding the gel or the liquid containing gel particles into aqueous medium Z1.

56. The method of claim 1, wherein aqueous medium Z1 is mixed in increments with the gel or the liquid containing gel particles in step (II), wherein the increments are up to about 100% of the weight of the gel or the liquid containing gel particles before the gel or the liquid is mixed with any aqueous medium Z1.

57. The method of claim 56, wherein the increments are up to about 80% of the weight of the gel or the liquid containing gel particles before the gel or the liquid is mixed with any aqueous medium Z1.

58. The method of claim 57, wherein the increments are up to about 60% of the weight of the gel or the liquid containing gel particles before the gel or the liquid is mixed with any aqueous medium Z1.

59. The method of claim 58, wherein the increments are up to about 40% of the weight of the gel or the liquid containing gel particles before the gel or the liquid is mixed with any aqueous medium Z1.

60. The method of claim 59, wherein the increments are up to about 20% of the weight of the gel or the liquid containing gel particles before the gel or the liquid is mixed with any aqueous medium Z1.

61. The method of claim 60, wherein the increments are up to about 10% of the weight of the gel or the liquid containing gel particles before the gel or the liquid is mixed with any aqueous medium Z1.

62. The method of claim 61, wherein the increments are up to about 5% of the weight of the gel or the liquid containing gel particles before the gel or the liquid is mixed with any aqueous medium Z1.

63. The method of claim 62, wherein the increments are up to about 1% of the weight of the gel or the liquid containing gel particles before the gel or the liquid is mixed with any aqueous medium Z1.

64. The method of claim 63, wherein the increments are up to about 0.5% of the weight of the gel or the liquid containing gel particles before the gel or the liquid is mixed with any aqueous medium Z1.

65. The method of claim 64, wherein the increments are up to about 0.1% of the weight of the gel or the liquid containing gel particles before the gel or the liquid is mixed with any aqueous medium Z1.

66. The method of claim 58, wherein the increments are from about 0.001% to about 10% of the weight of the gel or the liquid containing gel particles before the gel or the liquid is mixed with any aqueous medium Z1.

67. The method of claim 59, wherein the increments are from about 0.001% to about 5% of the weight of the gel or the liquid containing gel particles before the gel or the liquid is mixed with any aqueous medium Z1.

68. The method of claim 63, wherein the increments are from about 0.001% to about 1% of the weight of the gel or the liquid containing gel particles before the gel or the liquid is mixed with any aqueous medium Z1.

69. The method of claim 2, wherein aqueous medium Z1 is mixed in increments with the gel or the liquid containing gel particles in step (II), wherein the increments are up to about 100% of the weight of the gel or the liquid containing gel particles before the gel or the liquid is mixed with any aqueous medium Z1.

70. The method of claim 69, wherein the increments are up to about 80% of the weight of the gel or the liquid containing gel particles before the gel or the liquid is mixed with any aqueous medium Z1.

71. The method of claim 70, wherein the increments are up to about 60% of the weight of the gel or the liquid containing gel particles before the gel or the liquid is mixed with any aqueous medium Z1.

72. The method of claim 71, wherein the increments are up to 40% of the weight of the gel or the liquid containing gel particles before the gel or the liquid is mixed with any aqueous medium Z1.

73. The method of claim 72, wherein the increments are up to about 20% of the weight of the gel or the liquid containing gel particles before the gel or the liquid is mixed with any aqueous medium Z1.

74. The method of claim 73, wherein the increments are up to about 10% of the weight of the gel or the liquid containing gel particles before the gel or the liquid is mixed with any aqueous medium Z1.

75. The method of claim 74, wherein the increments are up to about 5% of the weight of the gel or the liquid containing gel particles before the gel or the liquid is mixed with any aqueous medium Z1.

76. The method of claim 75, wherein the increments are up to about 1% of the weight of the gel or the liquid containing gel particles before the gel or the liquid is mixed with any aqueous medium Z1.

77. The method of claim 76, wherein the increments are up to about 0.5% of the weight of the gel or the liquid containing gel particles before the gel or the liquid is mixed with any aqueous medium Z1.

78. The method of claim 77, wherein the increments are up to about 0.1% of the weight of the gel or the liquid containing gel particles before the gel or the liquid is mixed with any aqueous medium Z1.

79. The method of claim 71, wherein the increments are from about 0.001% to about 10% of the weight of the gel or the liquid containing gel particles before the gel or the liquid is mixed with any aqueous medium Z1.

80. The method of claim 72, wherein the increments are from about 0.001% to about 5% of the weight of the gel or the liquid containing gel particles before the gel or the liquid is mixed with any aqueous medium Z1.

81. The method of claim 76, wherein the increments are from about 0.001% to about 1% of the weight of the gel or the liquid containing gel particles before the gel or the liquid is mixed with any aqueous medium Z1.

82. The method of claim 2, wherein step (I) is conducted in the absence of any hydrating agent.

83. The method of claim 5, wherein step (I) is conducted in the absence of any hydrating agent.

84. The method of claim 10, wherein step (I) is conducted in the absence of any hydrating agent.

85. The method of claim 2, wherein in the gel or the liquid containing gel particles of step (I), the amount of the at least one liposome-forming lipid ranges from about 5% to about 95% by weight of the gel or the liquid containing gel particles.

86. The method of claim 85, wherein in the gel or the liquid containing gel particles of step (I), said amount of the at least one liposome-forming lipid ranges from about 10% to about 95% by weight of the gel or the liquid containing gel particles.

87. The method of claim 86, wherein in the gel or the liquid containing gel particles of step (I), said amount of the at least one liposome-forming lipid ranges from about 15% to about 95% by weight of the gel or the liquid containing gel particles.

88. The method of claim 87, wherein in the gel or the liquid containing gel particles of step (I), said amount of the at least one liposome-forming lipid ranges from about 20% to about 95% by weight of the gel or the liquid containing gel particles.

89. The method of claim 88, wherein in the gel or the liquid containing gel particles of step (I), said amount of the at least one liposome-forming lipid ranges from about 30% to about 95% by weight of the gel or the liquid containing gel particles.

90. The method of claim 89, wherein in the gel or the liquid containing gel particles of step (I), said amount of the at least one liposome-forming lipid ranges from about 40% to about 95% by weight of the gel or the liquid containing gel particles.

91. The method of claim 90, wherein in the gel or the liquid containing gel particles of step (I), said amount of the at least one liposome-forming lipid ranges from about 50% to about 95% by weight of the gel or the liquid containing gel particles.

92. The method of claim 2, wherein in the gel or the liquid containing gel particles of step (I), an amount of the at least one liposome-forming lipid ranges from about 5% to about 90% by weight of the gel or the liquid containing gel particles.

93. The method of claim 92, wherein in the gel or the liquid containing gel particles of step (I), said amount of the at least one liposome-forming lipid ranges from about 10% to about 90% by weight of the gel or the liquid containing gel particles.

94. The method of claim 93, wherein in the gel or the liquid containing gel particles of step (I), said amount of the at least one liposome-forming lipid ranges from about 15% to about 90% by weight of the gel or the liquid containing gel particles.

95. The method of claim 94, wherein in the gel or the liquid containing gel particles of step (I), said amount of the at least one liposome-forming lipid ranges from about 20% to about 90% by weight of the gel or the liquid containing gel particles.

96. The method of claim 95, wherein in the gel or the liquid containing gel particles of step (I), said amount of the at least one liposome-forming lipid ranges from about 30% to about 90% by weight of the gel or the liquid containing gel particles.

97. The method of claim 96, wherein in the gel or the liquid containing gel particles of step (I), said amount of the at least one liposome-forming lipid ranges from about 40% to about 90% by weight of the gel or the liquid containing gel particles.

98. The method of claim 97, wherein in the gel or the liquid containing gel particles of step (I), said amount of the at least one liposome-forming lipid ranges from about 50% to about 90% by weight of the gel or the liquid containing gel particles.

99. The method of claim 1, wherein at least one charged lipid is added in step (I) and the content of the at least one charged lipid in the gel or the liquid containing gel particles is from about 50% to about 100% of the weight of the lipid(s) in the gel or the liquid containing gel particles, wherein the at least one charged lipid is a lipid containing a net negative or positive charge, wherein the at least one charged lipid and the at least one liposome-forming lipid are the same or different.

100. The method of claim 99, wherein the at least one charged lipid is selected from the group consisting of N-acyl phosphatidylethanolamine, phosphatidylserine, phosphatidylinositol, phosphatidylglycerol, diphosphatidylglycerol and phosphatidic acid.

101. The method of claim 2, further comprising adding at least one charged lipid in step (I), wherein the content of the at least one charged lipid in the gel or the liquid containing gel particles is from about 50% to about 100% of the weight of the lipid(s) in the gel or the liquid containing gel particles, and wherein the at least one charged lipid is a lipid containing a net negative or positive charge, wherein the at least one charged lipid and the at least one liposome-forming lipid are the same or different.

102. The method of claim 101, wherein the at least one charged lipid is selected from the group consisting of N-acyl phosphatidylethanolamine, phosphatidylserine, phosphatidylinositol, phosphatidylglycerol, diphosphatidylglycerol and phosphatidic acid.

103. A method for preparing liposomes containing at least one biologically active substance encapsulated therein, said method comprising the following steps:
(I) (A) mixing at least one liposome-forming lipid, at least one charged lipid, the at least one biologically active substance, a water-miscible organic solvent and aqueous medium Y to form a gel or liquid containing gel particles without creation of any gas/aqueous phase boundary; or
(B) mixing at least one liposome-forming lipid, at least one charged lipid, a water-miscible organic solvent and aqueous medium Y to form a gel or liquid containing gel particles without creation of any gas/aqueous phase boundary;
(II) (A) mixing the gel or liquid containing gel particles of step (I)(A) with aqueous medium Z1 to directly form the liposomes containing the at least one biologically active substance encapsulated in the liposomes;
(B) (i) mixing the gel or liquid containing gel particles of step (I)(A) with aqueous medium Z1 to form a curd or curdy substance; and
(ii) mixing the curd or curdy substance with aqueous medium Z2 to directly form the liposomes containing the at least one biologically active substance encapsulated in the liposomes;
(C) (i) cooling the gel or liquid containing gel particles of step (I)(A) to form a waxy substance; and
(ii) mixing the waxy substance with aqueous medium Z1 to directly form the liposomes;
(D) mixing the gel or liquid containing gel particles of step (I)(B) with aqueous medium Z1 and the at least one biologically active substance to directly form the liposomes containing the at least one biologically active substance encapsulated in the liposomes;
(E) (i) mixing the gel or liquid containing gel particles of step (I)(B) with aqueous medium Z1 and the at least one biologically active substance to form a curd or curdy substance; and
(ii) mixing the curd or curdy substance with aqueous medium Z2 to directly form the liposomes containing the at least one biologically active substance encapsulated in the liposomes;
(F) (i) mixing the gel or liquid containing gel particles of step (I)(B) with aqueous medium Z1 to form a curd or curdy substance; and
(ii) mixing the curd or curdy substance with aqueous medium Z2 and the at least one biologically active substance to directly form the liposomes containing the at least one biologically active substance encapsulated in the liposomes; or
(G) (i) cooling the gel or liquid containing gel particles of step (I)(B) to form a waxy substance; and
(ii) mixing the waxy substance with aqueous medium Z1 and the at least one biologically active substance to directly form the liposomes;
wherein the at least one liposome-forming lipid and the at least one charged lipid are the same or different; wherein the content of the at least one charged lipid in the gel or liquid containing gel particles is about 50% to about 100% by weight of the lipid(s) in the gel or liquid containing gel particles; and wherein aqueous media Y, Z1 and Z2 are the same or different.

104. A method for preparing liposomes, said method comprising the following steps:
(I) mixing at least one liposome-forming lipid, a water-miscible organic solvent and aqueous medium Y to form a gel or liquid containing gel particles without sonication; wherein if the gel or liquid containing gel particles contains at least one acidic phospholipid, the content of the at least one acidic phospholipid is about 30% to about 100% by weight of the lipid(s) in the gel or liquid containing gel particles; and thereafter
(II) (a) mixing the gel or liquid containing gel particles with aqueous medium Z1 to directly form the liposomes;
(b) (i) mixing the gel or liquid containing gel particles with aqueous medium Z1 to form a curd or curdy substance; and
(ii) mixing the curd or curdy substance with aqueous medium Z2 to directly form the liposomes; or
(c) (i) cooling the gel or liquid containing gel particles to form a waxy substance; and
(ii) mixing the waxy substance with aqueous medium Z1 to directly form the liposomes;
wherein aqueous media Y, Z1 and Z2 are the same or different.

105. A method for preparing liposomes containing at least one biologically active substance encapsulated therein, said method comprising the following steps:
(I) (A) mixing at least one liposome-forming lipid, the at least one biologically active substance, a water-miscible organic solvent and aqueous medium Y to form a gel or liquid containing gel particles without sonication; wherein if the gel or liquid containing gel particles contains at least one acidic phospholipid, the content of the at least one acidic phospholipid is about 30% to about 100% by weight of the lipid(s) in the gel or liquid containing gel particles; or
(B) mixing at least one liposome-forming lipid, a water-miscible organic solvent and aqueous medium Y to form a gel or liquid containing gel particles without sonication; wherein if the gel or liquid containing gel particles contains at least one acidic phospholipid, the content of the at least one acidic phospholipid is about 30% to about 100% by weight of the gel or liquid containing gel particles;
(II) (A) mixing the gel or liquid containing gel particles of step (I)(A) with aqueous medium Z1 to directly form the liposomes containing the at least one biologically active substance encapsulated in the liposomes;
(B) (i) mixing the gel or liquid containing gel particles of step (I)(A) with aqueous medium Z1 to form a curd or curdy substance; and
(ii) mixing the curd or curdy substance with aqueous medium Z2 to directly form the liposomes containing the at least one biologically active substance encapsulated in the liposomes;
(C) (i) cooling the gel or liquid containing gel particles of step (I)(A) to form a waxy substance; and
(ii) mixing the waxy substance with aqueous medium Z1 to directly form the liposomes;
(D) mixing the gel or liquid containing gel particles of step (I)(B) with aqueous medium Z1 and the at least one biologically active substance to directly form the liposomes containing the at least one biologically active substance encapsulated in the liposomes;
(E) (i) mixing the gel or liquid containing gel particles of step (I)(B) with aqueous medium Z1 and the at least one biologically active substance to form a curd or curdy substance; and
(ii) mixing the curd or curdy substance with aqueous medium Z2 to directly form the liposomes containing the at least one biologically active substance encapsulated in the liposomes;
(F) (i) mixing the gel or liquid containing gel particles of step (I)(B) with aqueous medium Z1 to form a curd or curdy substance; and
(ii) mixing the curd or curdy substance with aqueous medium Z2 and the at least one biologically active substance to directly form the liposomes containing the at least one biologically active substance encapsulated in the liposomes; or
(G) (i) cooling the gel or liquid containing gel particles of step (I)(B) to form a waxy substance; and
(ii) mixing the waxy substance with aqueous medium Z1 and the at least one biologically active substance to directly form the liposomes;
wherein aqueous media Y, Z1 and Z2 are the same or different.

106. A method for preparing liposomes containing the at least one biologically active substance encapsulated therein comprising the following steps:
(i) (a) providing a gel or a liquid containing gel particles comprising at least one liposome-forming lipid, a water-miscible organic solvent, the least one biologically active substance and aqueous medium Y; or
(b) providing a gel or a liquid containing gel particles comprising at least one liposome-forming lipid, a water-miscible organic solvent, and aqueous medium Y; and thereafter
(ii) (a) mixing the gel or the liquid containing gel particles of step (i)(a) with aqueous medium Z to directly form the liposomes;
(b) (aa) mixing the gel or the liquid containing gel particles of step (i)(a) with aqueous medium Z to form a curd or curdy substance; and
(bb) mixing the curd or curdy substance with additional aqueous medium Z to directly form the liposomes, or
(c) (aa) cooling the gel or the liquid containing gel particles of step (i)(a) to form a waxy substance; and
(bb) mixing the waxy substance with aqueous medium Z to directly form the liposomes;
(d) mixing the gel or the liquid containing gel particles of step (i)(b) with the at least one biologically active substance and aqueous medium Z to directly form the liposomes;
(e) (aa) mixing the gel or the liquid containing gel particles of step (i)(b) with the at least one biologically active substance and aqueous medium Z to form a curd or curdy substance; and
(bb) mixing the curd or curdy substance with additional aqueous medium Z to directly form the liposomes; or
(f) (aa) cooling the gel or the liquid containing gel particles of step (i)(b) to form a waxy substance; and
(bb) mixing the waxy substance with the at least one biologically active substance and aqueous medium Z to directly form the liposomes;
wherein aqueous media Y and Z are the same or different.
